(19) United States
(12) Patent Application Publication
(10) Pub. No.: US 2021/0059544 A1
(43) Pub. Date: Mar. 4, 2021

(54) SENSOR MODULE AND BLOOD PRESSURE MEASUREMENT DEVICE

(71) Applicant: OMRON HEALTHCARE Co., Ltd., Kyoto (JP)

(72) Inventors: Yuki Nakagawa, Kyoto (JP); Iwanori Namba, Kyoto (JP); Takanobu Yamauchi, Kyoto (JP)

(21) Appl. No.: 17/098,979

(22) Filed: Nov. 16, 2020

Related U.S. Application Data
(63) Continuation of application No. PCT/JP2019/020047, filed on May 21, 2019.

(30) Foreign Application Priority Data
May 24, 2018 (JP) 2018-099731

(51) Int. Cl. A61B 5/022

(52) U.S. Cl. CPC A61B 5/02233; A61B 2562/046; A61B 2562/0247

(57) ABSTRACT

Provided is: a sensor module with a soft portion resistant to peeling off from the sensor head cover; and a blood pressure measurement device. A sensor module 63 includes a pressure sensor portion 71; a sensor head cover 73 including an opening 73a at a position opposite the pressure sensor portion 71; and a soft portion 74 that covers the pressure sensor portion 71 and is disposed in the opening 73a of the sensor head cover 73, a boundary portion between the soft portion 74 and the sensor head cover 73 including an outer edge portion 74b including an edge portion that is inclined or curved, when the sensor module is worn on a living body, with respect to a first direction along a circumferential direction of the living body or a second direction along a longitudinal direction of the living body.

### CROSS REFERENCE TO RELATED APPLICATIONS

This application is the U.S. national stage application filed pursuant to 35 U.S.C. 365(c) and 120 as a continuation of International Patent Application No. PCT/JP2019/020047, filed May 21, 2019 which application claims priority from Japanese Patent Application No. 2018-099731, filed May 24, 2018, which applications are incorporated herein by reference in their entireties.

### TECHNICAL FIELD

The present invention relates to a sensor module for measuring blood pressure and a blood pressure measurement device.

### BACKGROUND ART

In recent years, blood pressure measurement devices for measuring blood pressure are being used to monitor health status at home, as well as in medical facilities. In such blood pressure measurement devices, for example, known technologies using the oscillometric method and the tonometry method are used (see Patent Document 1, for example). A blood pressure measurement device using the oscillometric method detects vibration of the artery wall and measures blood pressure by using pressure sensor to detect the pressure of a cuff wrapped around the upper arm or wrist of a living body. A blood pressure measurement device using the tonometry method measures blood pressure by bringing a sensor module including a plurality of pressure sensors into contact with the wrist in a region of the wrist where the artery is found.

### CITATION LIST

### Patent Literature

Patent Document 1: JP H1-288228 A

### SUMMARY OF INVENTION

### Technical Problem

In the case in which a pressure sensor is disposed in a sensor module that comes into contact with skin, from the perspective of compatibility with a living body and protecting the pressure sensor, a configuration in which the pressure sensor is covered with resin or the like is employed. The sensor module, for example, is provided with: a sensor base installed with a pressure sensor, a sensor head cover that covers the sensor base and includes an opening at a position opposite the pressure sensor, and a soft portion formed from a soft resin that is disposed in the opening of the sensor head cover and covers the surface of the pressure sensor. The soft portion has a configuration in which the soft resin is filled in the opening formed in the sensor head cover, for example, and includes an outer edge with an identical shape to the opening edge of the sensor head cover. Generally, the opening of the sensor head cover has a rectangular shape, and the boundary between the sensor head cover and the soft portion is composed of two sets of parallel lines, one set being in a predetermined first direction and another set being in a second direction orthogonal to the first direction.

In such a blood pressure measurement device, for example, when the sensor module attached to the wrist or the like of a living body moves with respect to the living body, the surface of the sensor head cover and the soft portion slides across the living body while in contact with the living body. At this time, if the boundary line between the sensor head cover and the soft portion is orthogonal to the sliding direction, the join portion between the soft portion and the sensor head cover may peel off starting at the boundary line. The separating of join portion between the soft portion and the sensor head cover causes a decrease in waterproofness and causes dirt and the like to collect in the gap. The sensing accuracy may also be affected.

Thus, an object of the present invention is to provide: a sensor module with a soft portion resistant to peeling off from the sensor head cover; and a blood pressure measurement device.

### Solution to Problem

According to an aspect, provided is a sensor module including:

a sensor portion;
a sensor head cover including an opening at a position opposite the sensor portion; and
a soft portion that covers the sensor portion and is disposed in the opening of the sensor head cover, a boundary portion between the soft portion and the sensor head cover including an outer edge including an edge portion that is inclined or curved, when the sensor module is worn on a living body, with respect to a first direction along a circumferential direction of the living body or a second direction along a longitudinal direction of the living body.

According to this aspect, the boundary portion has a curved shape including an outer edge that includes an edge portion that is inclined or curved with respect to the first direction or the second direction. Thus, when movement relative to the living body occurs, a force received by the edge portion caused by friction with the living body can be reduced, and separation can be minimized or prevented. Thus, by preventing a gap from being formed between the soft portion and the opening, adhesion between the soft portion and the opening can be retained, waterproofing can be ensured, and dirt and the like collecting in the gap can be prevented. In addition, by preventing peeling off of the soft portion, negative effects on sensing accuracy can be minimized or prevented.

According to another aspect, provided is a sensor module, including:

a sensor portion;
a sensor base that supports the sensor portion;
a sensor head cover that is disposed opposite the sensor base and includes an opening at a position opposite the sensor portion; and
a soft portion that covers the sensor portion and is disposed in the opening of the sensor head cover, a boundary portion between the soft portion and the sensor head cover including an outer edge including an edge portion that is inclined or curved, when the sensor module is worn on a living body, with respect to a first direction along a circumferential direction of the living body or a second direction along a longitudinal direction of the living body.

According to this aspect, the boundary portion has a curved shape including an outer edge that includes an edge portion that is inclined or curved with respect to the first direction or the second direction. Thus, when movement relative to the living body occurs, a force in the direction of peeling off from the sensor base received by the edge portion caused by friction with the living body can be reduced, and separation can be minimized or prevented. Thus, by preventing a gap from being formed between the soft portion and the opening, adhesion between the soft portion and the opening can be retained, waterproofing can be ensured, and dirt and the like collecting in the gap can be prevented. In addition, by preventing peeling off of the soft portion, negative effects on sensing accuracy can be minimized or prevented.

A sensor module according to the sensor module of the aspect described above may be provided, wherein the outer edge of the soft portion is elliptical.

According to this aspect, because the outer edge of the boundary portion has an elliptical shape, resistance when moving can be dispersed with a simple configuration, and separation can be minimized or prevented.

A sensor module according to the sensor module of the aspect described above may be provided, wherein the edge portion is inclined with respect to the first direction and the second direction.

According to this aspect, because the boundary portion includes an inclined side, resistance when moving can be dispersed with a simple configuration, and separation can be minimized or prevented.

A sensor module according to the sensor module of the aspect described above may be provided, wherein the edge portion has a wave-like shape.

According to this aspect, because the length of the boundary portion is ensured and the adhesion area is ensured, the adhesive strength can be improved, and separation between the soft portion and the sensor head cover can be minimized or prevented.

A sensor module according to the sensor module of the aspect described above may be provided, wherein the boundary portion has a polygonal shape including an engagement portion increasing in width from a corner portion outward.

According to this aspect, with the configuration in which the polygonal shape includes the engagement portion increasing in width from the corner portion outward and a structure in which the sensor head cover fits into the base end portion of the engagement portion, the soft portion falling out can be prevented, and separation between the soft portion and the sensor head cover can be minimized or prevented.

According to another aspect, provided is a sensor module including:

a sensor portion;
a sensor base that supports the sensor portion;
a sensor head cover that is disposed opposite the sensor base and includes an opening at a position opposite the sensor portion; and
a soft portion disposed in the opening of the sensor head cover, the soft portion having a polygonal shape that covers the sensor portion and including an engagement projection increasing in width from a corner portion outward.

According to this aspect, with the configuration including a circular engagement portion at the corner portion and a structure in which a portion of the opening edge of the sensor head cover fits into the base end portion of the engagement portion, the soft portion falling out can be prevented, and separation between the soft portion and the sensor head cover can be minimized or prevented.

A sensor module according to the sensor module of the aspect described above may be provided, wherein

the sensor head cover is provided with a step portion in a circumferential edge portion of the opening at an end surface on one side, the step portion being recessed toward the other side; and
a surface of the step portion on the one side is covered by the soft portion.

According to this aspect, separation between the soft portion and the sensor head cover can be minimized or prevented with a simple configuration.

A sensor module according to the sensor module of the aspect described above may be provided, wherein the sensor head cover is provided, at a circumferential edge portion of the opening, with a wall portion that covers one side of the soft portion.

According to this aspect, with a structure in which the end surface of the engagement portion is closed off by the wall portion, the soft portion at the engagement portion falling off can be prevented, and separation between the soft portion and the sensor head cover can be minimized or prevented.

A sensor module according to the sensor module of the aspect described above may be provided, wherein

the sensor portion is provided with a pressure sensitive element array in which a plurality of pressure sensitive elements are arranged in the first direction;
and
an end portion in the second direction of the outer edge of the boundary and the pressure sensitive element array are separated in the second direction.

According to this aspect, when the sensor module moves in the longitudinal direction of the living body relatively thereto, even if the soft portion separates, because there is distance to the pressure sensitive element arrays, peeling off affecting the sensing portion can be prevented.

A sensor module according to the sensor module of the aspect described above may be provided, wherein

the sensor base includes: a pressure sensitive element array including a plurality of pressure sensitive elements disposed on one main surface side and a support wall portion in which a flow hole is disposed in an outer circumference of a region where the pressure sensitive element array is disposed, the flow hole extending through from the one main surface side to an other main surface side;
the sensor head cover is disposed on the one main surface side of the support wall portion of the sensor base via a gap that communicates with the flow hole and the opening; and
the soft portion is formed in the opening.

According to this aspect, the soft portion can be easily formed, and separation between the soft portion and the sensor head cover can be minimized or prevented.

According to another aspect, provided is a blood pressure measurement device, including:

any one of the sensor modules described above;
a sensing body including a case that houses the sensor module;
an attach portion including: an opening portion disposed at a position opposite a region where one artery of a wrist is present, the opening portion having a shape that allows the wrist to be palpated and an end surface that curves conforming to a portion of a shape in the circumferential direction of the wrist; and
a fastener provided on the attach portion that extends in a first direction and is wrapped around in the circumferential direction of the wrist.

According to this aspect, when the sensor module moves in the circumferential direction of the wrist and in the length direction orthogonal thereto, the soft portion can be prevented from separating from the sensor or the cover due to the movement.

### Advantageous Effects of Invention

The present invention can provide: a sensor module with a soft portion resistant to peeling off from a sensor head cover; and a blood pressure measurement device.

### BRIEF DESCRIPTION OF DRAWINGS

FIG. 1 is a perspective view illustrating the configuration of a blood pressure measurement device according to an embodiment of the present invention.

FIG. 2 is a block diagram illustrating the configuration of the blood pressure measurement device.

FIG. 3 is a perspective view illustrating the configuration of a sensor device of the blood pressure measurement device.

FIG. 4 is a perspective view illustrating the configuration of a portion of the sensor device of the blood pressure measurement device.

FIG. 5 is a perspective view illustrating the configuration of a sensor unit of the blood pressure measurement device.

FIG. 6 is a plan view illustrating the configuration of the sensor unit.

FIG. 7 is a cross-sectional view illustrating the configuration of a sensor module and an air bag of the sensor unit.

FIG. 8 is a cross-sectional view illustrating the configuration of the sensor module and the air bag of the sensor unit.

FIG. 9 is a cross-sectional view illustrating the configuration of the sensor module and the air bag of the sensor unit.

FIG. 10 is a cross-sectional view illustrating the configuration of the blood pressure measurement device in a state of being attached to a wrist.

FIG. 11 is a cross-sectional view illustrating the configuration of the blood pressure measurement device in a state of being attached to the wrist.

FIG. 12 is a cross-sectional view illustrating the configuration of the blood pressure measurement device in a state of being attached to the wrist.

FIG. 13 is a cross-sectional view illustrating the configuration of the sensor module of the sensor unit.

FIG. 14 is a cross-sectional view illustrating the configuration of the sensor module.

FIG. 15 is an explanatory diagram illustrating the configuration of a portion of the sensor module.

FIG. 16 is a plan view illustrating the configuration of the sensor module of the sensor unit.

FIG. 17 is a perspective view illustrating the configuration of a portion of the sensor module.

FIG. 18 is a plan view illustrating the configuration of the sensor module.

FIG. 19 is an explanatory diagram illustrating an example of a method for manufacturing the sensor module.

FIG. 20 is an explanatory diagram illustrating a filling step of a method for manufacturing the sensor module.

FIG. 21 is a perspective view illustrating the configuration of a portion of the sensor module of the blood pressure measurement device.

FIG. 22 is an explanatory diagram illustrating the position adjustment of the sensor unit of the blood pressure measurement device.

FIG. 23 is a flowchart illustrating an example of blood pressure measurement using the blood pressure measurement device.

FIG. 24 is an explanatory diagram illustrating an example of blood pressure measurement using the blood pressure measurement device.

FIG. 25 is an explanatory diagram illustrating an example of blood pressure measurement using the blood pressure measurement device.

FIG. 26 is a plan view illustrating the configuration of a sensor module of a blood pressure measurement device according to another embodiment of the present invention.

FIG. 27 is a plan view illustrating the configuration of a sensor module of a blood pressure measurement device according to another embodiment of the present invention.

FIG. 28 is a plan view illustrating the configuration of a sensor module of a blood pressure measurement device according to another embodiment of the present invention.

FIG. 29 is a plan view illustrating the configuration of a sensor module of a blood pressure measurement device according to another embodiment of the present invention.

FIG. 30 is a plan view illustrating the configuration of a sensor module of a blood pressure measurement device according to another embodiment of the present invention.

FIG. 31 is a plan view illustrating the configuration of a sensor module of a blood pressure measurement device according to another embodiment of the present invention.

FIG. 32 is a plan view illustrating the configuration of a sensor module of a blood pressure measurement device according to another embodiment of the present invention.

FIG. 33 is a perspective view illustrating the configuration of the sensor module of the blood pressure measurement device according the embodiment.

FIG. 34 is a plan view illustrating the configuration of a sensor module of a blood pressure measurement device according to another embodiment of the present invention.

FIG. 35 is a perspective view illustrating the configuration of a blood pressure measurement device according to another embodiment of the present invention.

FIG. 36 is a block diagram illustrating the configuration of the blood pressure measurement device.

FIG. 37 is a perspective view illustrating the configuration of a blood pressure measurement device according to another embodiment of the present invention.

### DESCRIPTION OF EMBODIMENTS

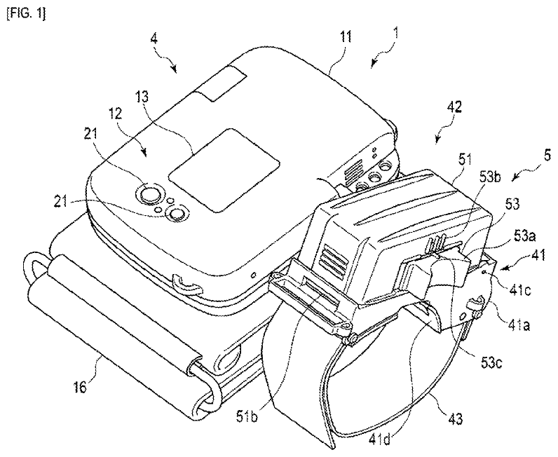

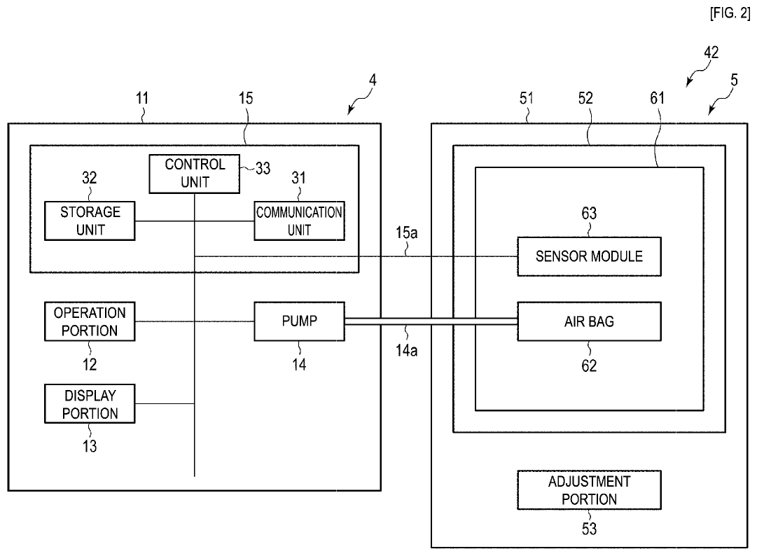

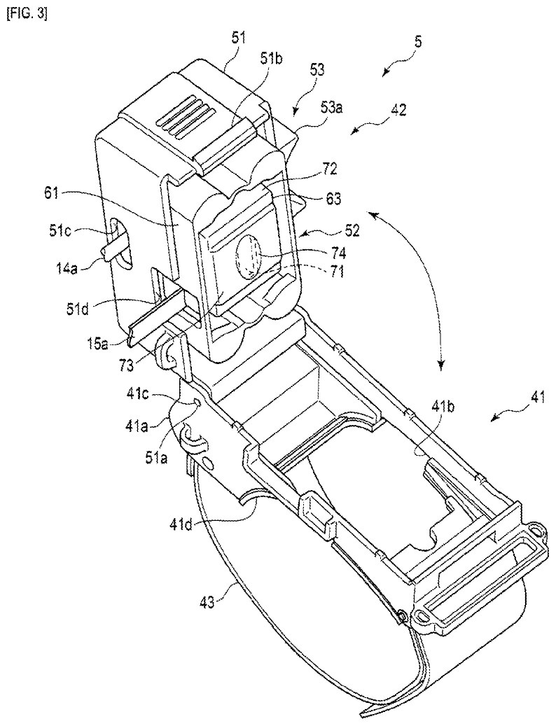

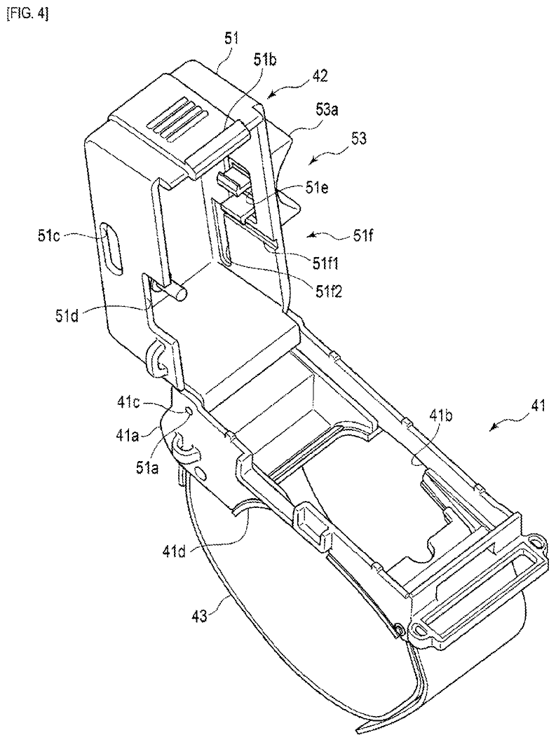

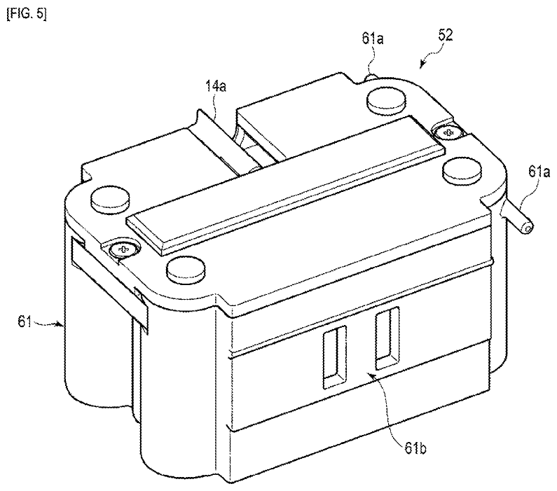

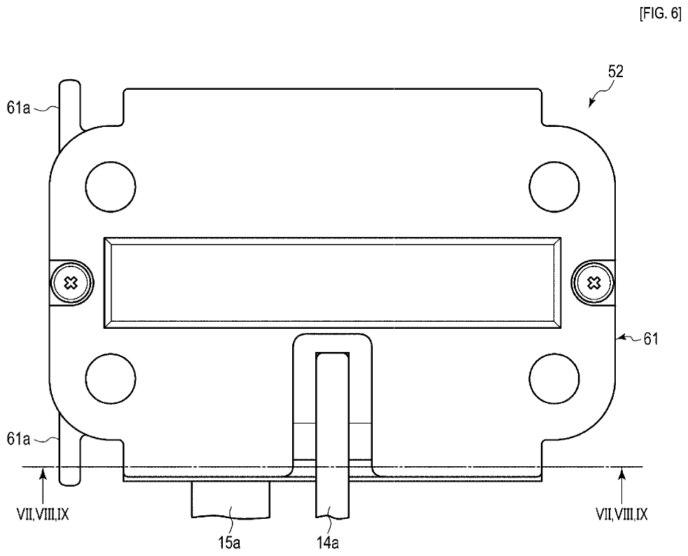

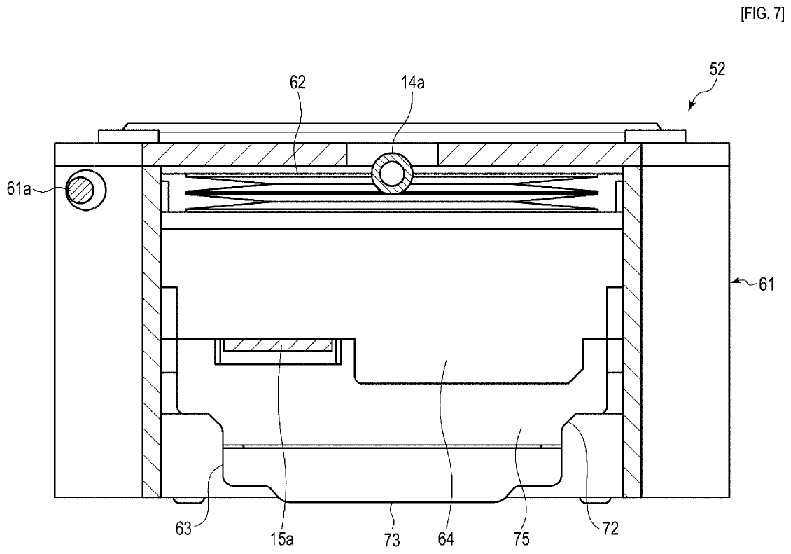

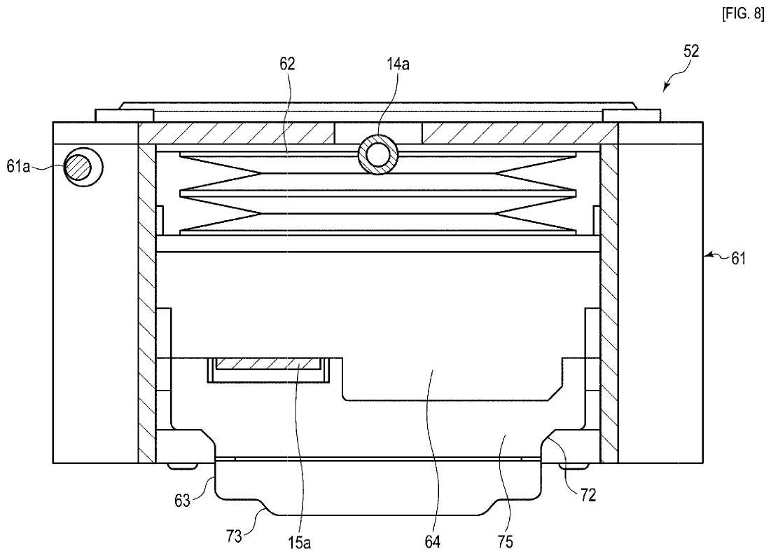

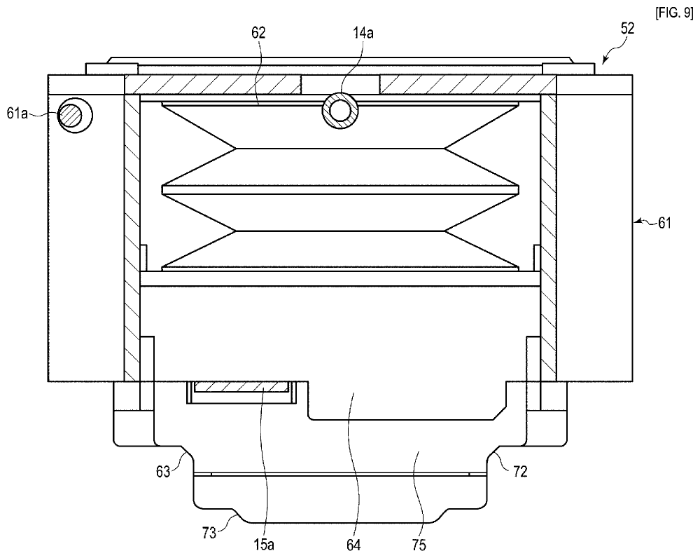

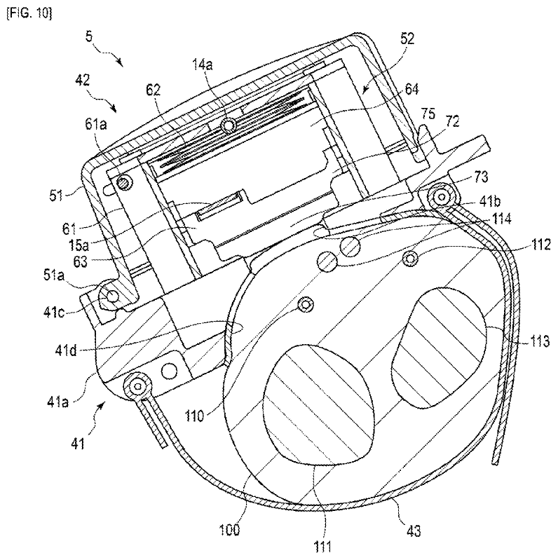

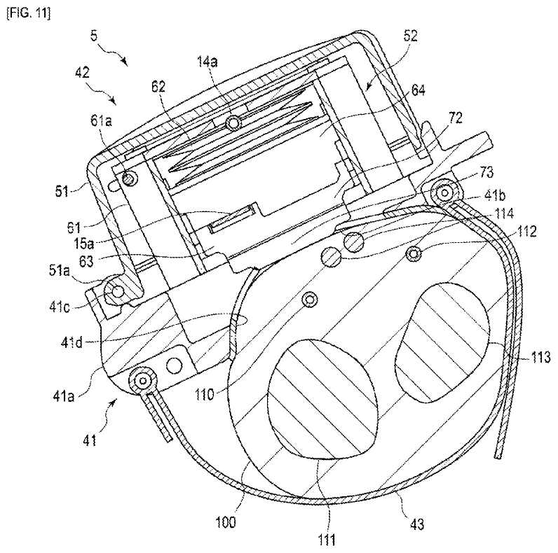

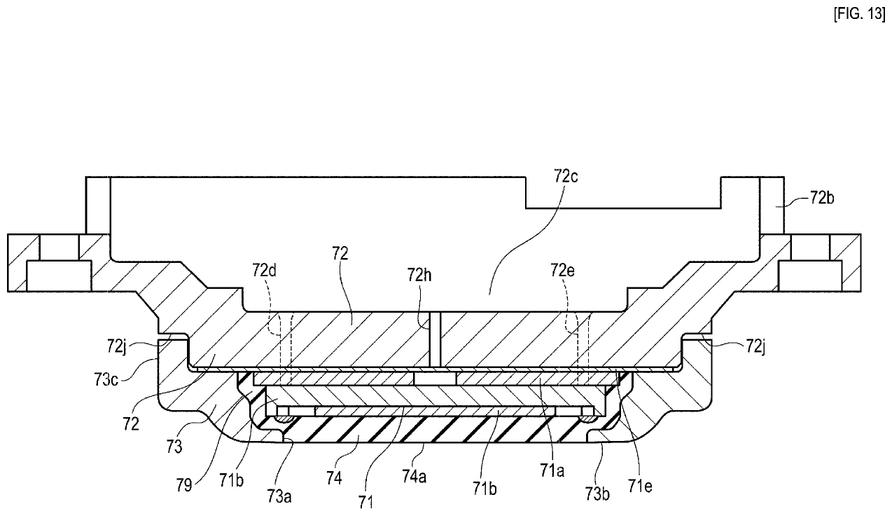

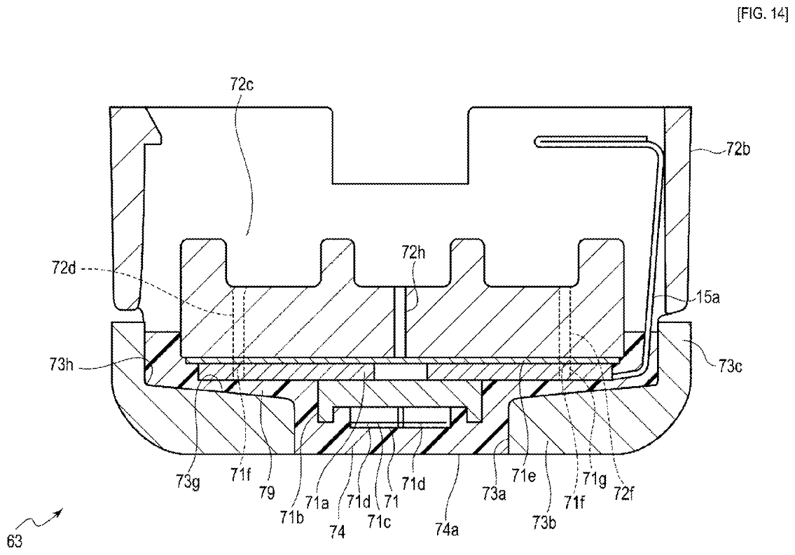

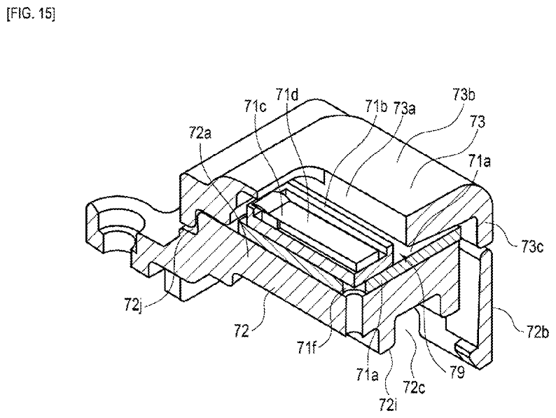

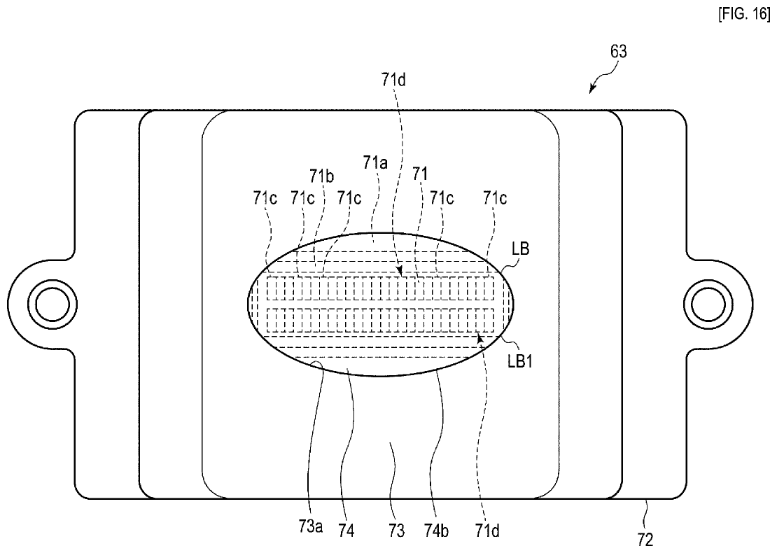

FIG. 1 is a perspective view illustrating the configuration of the blood pressure measurement device 1 according to an embodiment of the present invention in a state in which a body fastener 16 is closed. FIG. 2 is a block diagram illustrating the configuration of the blood pressure measurement device 1. FIG. 3 is a perspective view illustrating the configuration of a sensor device 5 of the blood pressure measurement device 1 in a state in which a sensing body 42 is open. FIG. 4 is a perspective view illustrating the configuration of the blood pressure measurement device 1 with a sensor unit 52 removed from the sensor device 5. FIG. 5 is a perspective view illustrating the configuration of the sensor unit 52 of the blood pressure measurement device 1. FIG. 6 is a plan view illustrating the configuration of the sensor unit 52. FIG. 7 is a cross-sectional view illustrating the configuration of a sensor module 63 and an air bag 62 of the sensor unit 52 taken along a cross-section line VII-VII in FIG. 6. FIG. 8 is a cross-sectional view illustrating the configuration of the sensor module 63 and the air bag 62 taken along a cross-section line VIII-VIII in FIG. 6. FIG. 9 is a cross-sectional view illustrating the configuration of the sensor module 63 and the air bag 62 taken along a cross-section line IX-IX in FIG. 6. FIGS. 10 to 12 are cross-sectional views illustrating the configuration of the blood pressure measurement device 1 in a state of being attached to a wrist 100. FIGS. 13 and 14 are cross-sectional views illustrating the configuration of the sensor module 63 of the sensor unit 52. FIG. 15 is an explanatory diagram illustrating the configuration of a portion of the sensor module 63. FIG. 16 is a plan view illustrating the configuration of the sensor module of the sensor unit.

Note that in the drawings, a radial artery of a wrist 100 is denoted as 110, a radius is denoted as 111, an ulnar artery is denoted as 112, an ulna is denoted as 113, and a tendon is denoted as 114.

The blood pressure measurement device 1 is an electronic blood pressure measurement device that is attached to the wrist 100 of a living body and calculates a blood pressure value from the pressure of the radial artery 110. As illustrated in FIGS. 1 to 15, the blood pressure measurement device 1 includes a device body 4 and the sensor device 5. For example, the blood pressure measurement device 1 has a configuration in which the sensor device 5 is attached to a region of the wrist 100 where the radial artery 110 is found and in which the device body 4 is attached to the wrist 100 adjacent to the sensor device 5 on the elbow side.

The blood pressure measurement device 1, by pressing the radial artery 110 with the sensor device 5, measures the pressure of the pressure pulse wave per heart beat that changes in conjunction with the heart rate of the radial artery 110, executes, via the device body 4, processing based on the tonometry method on the measured pressure, and obtains the blood pressure.

As illustrated in FIGS. 1 and 2, the device body 4 includes: a body case 11, an operation portion 12, a display portion 13, a pump 14, a control board 15, and the body fastener 16. Also, for example, the device body 4 may be provided with a cuff on the body fastener 16 that is configured to compress the wrist 100 during blood pressure measurement.

The body case 11 houses: a portion of the operation portion 12, a portion of the display portion 13, and the control board 15 and exposes: a portion of the operation portion 12 and a portion of the display portion 13 from the outer surface. In addition, the body fastener 16 is attached to the body case 11.

The operation portion 12 is configured to receive an instruction input from a user. For example, the operation portion 12 includes: a plurality of buttons 21 provided on the body case 11 and a sensor that detects operation of the buttons 21. Note that the operation portion 12 may be provided on the display portion 13 as a touch panel. When operated by the user, the operation portion 12 converts an instruction into an electrical signal. The sensor that detects operation of the buttons 21 is electrically connected to the control board 15 and outputs an electrical signal to the control board 15.

The display portion 13 is disposed in the body case 11 and is exposed from the outer surface of the body case 11. The display portion 13 is electrically connected to the control board 15. The display portion 13 is, for example, a liquid crystal display or an organic electroluminescent display. The display portion 13 displays various information including measurement results such as date and time; blood pressure values like maximum blood pressure and minimum blood pressure; heart rate; and the like.

The pump 14 is, for example, a piezoelectric pump. The pump 14 includes a tube 14a connected to the sensor device 5 for compressing air and supplying compressed air to the sensor device 5 via the tube 14a. The pump 14 is electrically connected to the control board 15.

As illustrated in FIG. 2, the control board 15 includes a communication unit 31, a storage unit 32, and a control unit 33, for example. The control board 15 is configured by the communication unit 31, the storage unit 32, and the control unit 33 being mounted on the board. Also, the control board 15 is connected to the sensor device 5 via a cable 15a. The cable 15a runs from inside the body case 11 to outside the body case 11 via a portion of the outer surface of the body case 11. For example, the cable 15a runs from inside the body case 11 to the sensor device 5 via an opening formed in a side surface of the body case 11.

The communication unit 31 is configured to transmit and receive information from an external device wirelessly or via a wire. The communication unit 31 transmits information, such as information controlled by the control unit 33, measured blood pressure values, pulse, and the like, to an external device via a network and receives a program for software update or the like from an external device via a network and sends this to the control unit.

In the present embodiment, the network is, for example, the Internet, but no such limitation is intended. The network may be a network such as a Local Area Network (LAN) provided in a hospital or may be a direct wired communication with an external device, using a cable or the like including terminals of a predetermined protocol such as USB. Thus, the communication unit 31 may include a plurality of wireless antennas, micro-USB connectors, or the like.

The storage unit 32 pre-stores: program data for controlling the entire blood pressure measurement device 1; settings data for configuring various functions of the blood pressure measurement device 1; calculation data for calculating blood pressure values and pulse from the pressure measured by the pressure sensitive elements 71c; and the like. Furthermore, the storage unit 32 stores information such as: the calculated blood pressure value; pulse; time series data in which this calculated data and time are associated; and the like.

The control unit 33 is composed of, for example, a single or a plurality of central processing units (CPU), controls the operation of the entire blood pressure measurement device 1, and executes each processing on the basis of the program data. The control unit 33 is electrically connected to the operation portion 12, the display portion 13, the pump 14, and the sensor device 5, controls the operation of each configuration, transmits and receive signals, and supplies power.

The body fastener 16 includes, for example, one or a plurality of band-like bands; and a fixing member such as a hook-and-loop fastener that secures the band wrapped around the wrist 100. The body fastener 16 fixes the body case 11 to the wrist 100.

With the device body 4 having such a configuration, by the control unit 33 executing processing using the program data stored in the storage unit 32, blood pressure data can be continuously generated from the pulse waves of the radial artery 110 detected by the sensor device 5. The blood pressure data includes data of blood pressure waveforms corresponding to the waveforms of measured pulse waves. The blood pressure data may further include time series data of a blood pressure feature value (blood pressure value). The blood pressure feature value includes, for example and without limitation, systolic blood pressure (SBP) and diastolic blood pressure (DBP). The maximum value in the pulse wave waveform per heart beat corresponds to systolic blood pressure, and the minimum value in the pulse wave waveform of per heart beat corresponds to diastolic blood pressure.

In this embodiment, the device body 4 measures the pressure pulse wave as a pulse wave by the tonometry method. Here, the tonometry method refers to a method for pressing the radial artery 110 from above the skin with appropriate pressure, forming a flat portion in the artery, and measuring the pressure pulse wave with the sensor device 5 in a balanced state between the interior and the exterior of the radial artery 110. According to the tonometry method, a blood pressure value per heart beat can be acquired.

As illustrated in FIGS. 1, 3, and 4, the sensor device 5 includes: an attach portion 41, the sensing body 42, and a fastener 43.

The attach portion 41 includes a main surface that has a shape that conforms to the circumferential direction of the wrist 100 in the region where the radial artery 110 of the left wrist 100 is found. As a specific example, the attach portion 41 includes a base portion 41a that curves conforming to the shape in the circumferential direction of the region in contact with the wrist 100; an opening portion 41b formed in the base portion 41a, an attachment portion 41c provided on the base portion 41a for attaching the sensing body 42; and a cushion 41d provided on a main surface of the base portion 41a that comes into contact with the wrist 100.

The base portion 41a is configured to be elongated in one direction. The base portion 41a is disposed on a palm side of wrist 100 and on a side portion side on the radius 111 side of the wrist 100, and the main surface disposed on the wrist 100 side curves conforming to the shape in the circumferential direction of the palm side of the wrist 100 and the side portion side on the radius 111 side of the wrist 100. Furthermore, at least the outer circumferential edge side of the main surface of the base portion 41a comes into contact with the sensing body 42.

The opening portion 41b is provided in a central region of the base portion 41a and is formed with a size of one or a plurality of fingers. That is, the opening portion 41b is formed with a size that allows the region where the radial artery 110 of the wrist 100 is exposed from the opening portion 41b to be palpated by a finger, when the sensor device 5 is attached to the wrist 100, and that allows a portion of the sensing body 42 to come into contact with the wrist 100.

The attachment portion 41c is provided on a main surface of the base portion 41a opposite the surface facing the wrist 100 and provided on an end side of the base portion 41a in the longitudinal direction. The attachment portion 41c supports the sensing body 42 and is configured to move the sensing body 42 in a direction away from the base portion 41a and a direction toward the base portion 41a. As a specific example, the attachment portion 41c is a journal-like portion that rotatably journals the sensing body 42 about an axis. For example, the attachment portion 41c is integrally formed with the base portion 41a.

The cushion 41d is, for example, an elastic body configured in a sheet shape from a foaming resin material provided on a main surface of the base portion 41a that comes into contact with the wrist 100. The cushion 41d protects wrist 100 by elastically deforming, for example, when the blood pressure measurement device 1 is worn on the wrist 100.

As illustrated in FIGS. 2 to 12, the sensing body 42 includes: a case 51, the sensor unit 52, and an adjustment portion 53 for adjusting the position of the sensor unit 52.

The case 51 has a rectangular box shape with an open surface opposite the attach portion 41, for example. The case 51 supports the sensor unit 52 and the adjustment portion 53. Furthermore, the case 51 is attached to the attachment portion 41c in a manner to be movable back and forth in a direction away from the base portion 41a. As a specific example, the case 51 includes a rotation shaft 51a rotatably disposed in the attachment portion 41c. Also, the case 51 includes an engagement portion 51b that fixes the case 51 to the base portion 41a when it comes into contact with the base portion 41a. The engagement portion 51b, for example, is a projection that engages with an opening provided on the base portion 41a and, by being operated, is configured to release the engagement with the opening of the base portion 41a.

Furthermore, the case 51 includes: a first hole portion 51c where the tube 14a is disposed, a second hole portion 51d where the cable 15a is disposed, a third hole portion 51e that movably supports a portion of the adjustment portion 53, and a guide groove 51f that guides the movement of the sensor unit 52.

The first hole portion 51c and the second hole portion 51d are provided on the same side wall of the case 51 adjacent to the device body 4 when the device is worn on the wrist 100.

The third hole portion 51e is provided on a side wall opposite to the side wall of the case 51 where the first hole portion 51c and the second hole portion 51d are provided. The third hole portion 51e is a rectangular opening that linearly extends in the longitudinal direction of the case 51, or in other words, the circumferential direction of the wrist 100 when the sensor device 5 is attached to the wrist 100.

The guide groove 51f is provided on the inner surface side of the side wall of the case 51 provided with the third hole portion 51e. The guide groove 51f includes: a first groove 51f1 that extends from an opening end portion of the case 51 to partway toward the ceiling opposite the opening; and a second groove 51f2 that extends in a direction orthogonal to the first groove 51f1. The second groove 51f2 connects to the first groove 51f1 at one end and extends from this end to the other end toward one side in the longitudinal direction of the case 51.

The sensor unit 52 includes: a movable case 61, the air bag 62, the sensor module 63, and a movable base 64 that supports the sensor module 63 to be movable in one direction with respect to the movable case 61. The sensor unit 52 is supported by the case 51 in a manner to be movable in a predetermined range in the longitudinal direction of the case 51 via the adjustment portion 53.

The movable case 61 houses the sensor module 63 and the movable base 64 and supports the movable base 64 supporting the sensor module 63 in a manner allowing the movable base 64 to be movable toward the opening portion 41b of the attach portion 41. The movable case 61 is supported in a manner to be movable in the longitudinal direction of the case 51 inside the case 51.

As a specific example, the movable case 61 has a rectangular box shape with the surface opposite the attach portion 41 housing the air bag 62 and the sensor module 63 being open. The movable case 61 houses the air bag 62, the sensor module 63, and the movable base 64. In the movable case 61, the air bag 62 is disposed between the ceiling, the sensor module 63, and the movable base 64. The movable case 61 supports the movable base 64 in a manner allowing the movable base 64 to be movable in one direction so that the sensor module 63 can protrude out from the opening of the movable case 61.

The movable case 61 includes: a guide projection 61a disposed on the outer surface of a side wall opposite the side wall on which the guide groove 51f of the case 51 is provided in a manner allowing the guide projection 61a to move in the guide groove 51f; and a fixing portion 61b in which a portion of the adjustment portion 53 is fixed. As the guide projection 61a moves in the second groove 51f2, the movable case 61 moves in the longitudinal direction of the case 51.

The air bag 62 has a bellows-like structure. The air bag 62 is fluidly connected to the pump 14 via the tube 14a. As illustrated in FIGS. 7 to 12, the air bag 62 expands in a direction from the ceiling of the movable case 61 toward the opening. When the air bag 62 expands, the sensor module 63 is moved from a position where the sensor module 63 is housed within the movable case 61 to a position where the sensor module 63 projects from the opening of the movable case 61 and comes into contact with the wrist 100 via the opening portion 41b of the attach portion 41. The air bag 62 is formed from polyurethane, for example. The air bag 62 and the pump 14 together constitute a pressing mechanism that presses the sensor module 63 toward the wrist.

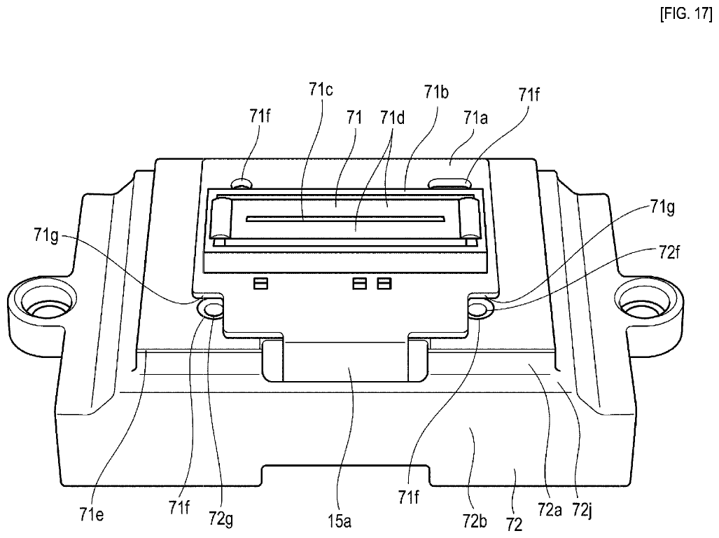

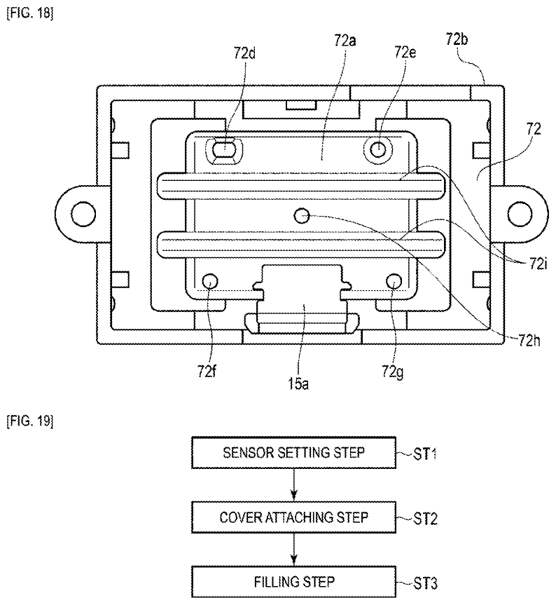

As illustrated in FIGS. 13 to 18, the sensor module 63 includes: the pressure sensor portion 71, the sensor base 72 that supports the pressure sensor portion 71, a sensor head cover 73 that covers the sensor base 72 and includes an opening 73a in a region opposite the pressure sensor portion 71, and a soft portion 74 provided in the opening 73a of the sensor head cover 73. FIG. 17 is a perspective view illustrating the configuration of a portion of the sensor module 63. FIG. 18 is a plan view of the sensor module 63 as viewed from another side.

The sensor module 63 is disposed inside the movable case 61 and is supported by the movable case 61 in a manner allowing the sensor module 63 to move in a predetermined movement range in the direction of the ceiling and the opening of the movable case 61 opposing one another. In other words, the sensor module 63 is supported in a manner to be movable within the movable case 61, and the movement is restricted by a restriction portion such as a stopper or like when the sensor module 63 moves from the opening of the movable case 61 to the position where the sensor module 63 projects out a certain amount or more.

The pressure sensor portion 71 includes: a flexible substrate 71a, a substrate 71b mounted on the flexible substrate 71a, and a plurality of pressure sensitive elements 71c mounted on the substrate 71b. The pressure sensor portion 71 is mounted on one main surface of the sensor base 72 and transmits a pressure value measured by the plurality of pressure sensitive elements 71c to the control board 15 via the cable 15a.

The flexible substrate 71a has a rectangular plate-like shape and is adhered on the sensor base 72 via an adhesive sheet 71e. The flexible substrate 71a and the adhesive sheet 71e are provided with an opening 71f and a cutout portion 71g formed at positions overlapping flow holes 72d, 72e, 72f, 72g of the sensor base 72 described below and are formed with a shape that does not close off the flow holes 72d, 72e, 72f, 72g.

A predetermined circuit pattern and the substrate 71b are formed on one main surfaces of the flexible substrate 71a. The flexible substrate 71a is connected to the cable 15a and is electrically connected to the control board 15 via the cable 15a.

The substrate 71b has a rectangular plate-like shape and supports the plurality of pressure sensitive elements 71c. The substrate 71b and the plurality of pressure sensitive elements 71c constitute a sensor chip.

The plurality of pressure sensitive elements 71c (the pressure sensor) are arranged in one direction, forming a pressure sensitive element array 71d. The plurality of pressure sensitive elements 71c are arranged side by side in one direction along the width direction of the wrist 100 when the device is worn, for example. A single or a plurality of the pressure sensitive element arrays 71d are provided. In the case in which the plurality of the pressure sensitive element arrays 71d are provided, the plurality of pressure sensitive element arrays 71d are disposed at predetermined intervals in a direction orthogonal to the arrangement direction of the plurality of pressure sensitive elements 71c. In the present embodiment, two rows of the pressure sensitive element arrays 71d are disposed. The pressure sensitive elements 71c are electrically connected to the circuit on the flexible substrate 71a.

The sensor base 72 is made from a synthetic resin, for example, includes, integrally, a support wall portion 72a that supports the pressure sensor portion 71 and a circumferential wall portion 72b vertically provided around the outer circumferential edge of the support wall portion 72a on the rear surface side on the opposite side to the living body, and is provided with a recess portion 72c on the rear side of the support wall portion 72a. The sensor base 72 supports the pressure sensor portion 71 and the cable 15a connected to the pressure sensor portion 71.

The support wall portion 72a has a rectangular plate-like shape with a predetermined thickness. The support wall portion 72a, on the surface side, which is the living body side, supports the pressure sensor portion 71 in a region opposite the opening 73a of the sensor head cover 73. A step portion 72j with a projecting central portion is formed in an outer circumferential edge portion of the support wall portion 72a. When the sensor head cover 73 and the sensor base 72 are assembled together, a frame portion 73c engages with the step portion 72j and is placed in position.

The support wall portion 72a includes a plurality of through-holes that extend through in the thickness direction. Specifically, on either side of the pressure sensor portion 71, the first hole 72d and the second hole 72e forming a flow entrance are located in a side portion on one side, and the third hold 72f and the fourth hole 72g forming a flow entrance are located in a side portion on the other side. In other words, the flow holes 72d, 72e, 72f, 72g are disposed on both side portions on either side of the pressure sensitive element arrays 71d.

In the side portion of the support wall portion 72a on one side of the pressure sensor portion 71, the first hole 72d is located at an end in the longitudinal direction of the pressure sensor portion 71, and the second hole 72e is located at the other end. Also, in the side portion of the support wall portion 72a on the other side of the pressure sensor portion 71, the third hole 72f is formed at an end in the longitudinal direction of the pressure sensor portion 71, and the fourth hole 72g is formed at the other end.

A fifth hole 72h is a circular hole that extends through in the thickness direction located in a central portion of the support wall portion 72a where the pressure sensor portion 71 is disposed.

The circumferential wall portion 72b is erected up from the outer circumference of the support wall portion 72a on the opposite side to the living body, and the recess portion 72c that opens to the rear surface side of the sensor base 72 is formed by the support wall portion 72a and the circumferential wall portion 72b. Recesses and protrusions are formed along the first direction in the rear surface of the support wall portion 72a. Specifically, ridge portions 72i that extend in the first direction along the longitudinal direction of the pressure sensor portion 71 are formed on the rear surface of the support wall portion 72a in a region in both side portions on either side of the region where the pressure sensor portion 71 is disposed.

The sensor head cover 73 is formed from a synthetic resin, for example, and has a rectangular shape with a central portion projecting to the living body side. The sensor head cover 73 includes, integrally, a protrusion portion 73b including the opening 73a and the frame portion 73c disposed on the circumferential edge of the protrusion portion 73b.

The protrusion portion 73b has a plate-like shape including the rectangular opening 73a. At least the central portion of the main surface that comes into contact with the living body on one side of the protrusion portion 73b is formed flat. The main surface on the other side of the protrusion portion 73b has a tapered shape including an inclined surface 73g that inclines to one side toward the opening 73a centrally located. Moreover, the inner wall of a corner portion 73h, which is the boundary between the protrusion portion 73b and the frame portion 73c is rounded by surface processing and formed with a curved surface. The sensor head cover 73 has a configuration having no corners, from the inner wall of the frame portion 73c positioned on the outer circumference, along the curved surface of the corner portion 73h, to the opening 73a. A gap portion 79 between the sensor head cover 73 and the sensor base 72 is formed with low fluid resistance.

The frame portion 73c is erected from the outer circumferential edge of the protrusion portion 73b on the sensor base 72 side and engages with the step portion 72j of the outer circumferential edge of the support wall portion 72a of the sensor base 72.

At least a portion of the opposing surfaces of the sensor head cover 73 and the sensor base 72 are separated from one another, and the gap portion 79 is formed between the inner surface of the sensor head cover 73 and the outer surface of the sensor base 72. The pressure sensor portion 71 and the soft portion 74 are disposed in the gap portion 79.

In the present embodiment, the gap portion 79 is formed, as a gap, between the surface on one side of the support wall portion 72a on which the pressure sensor portion 71 is mounted and the surface on the other side of the protrusion portion 73b, and the gap portion 79 is formed between the outer circumferential surface of the support wall portion 72a and the inner circumferential surface of the frame portion.

The gap portion 79 communicates with the plurality of flow holes 72d to 72g of the sensor base 72 through the cutout portion 71g of the flexible substrate 71a and the opening 71f of the adhesive sheet 71e. In other words, the gap portion 79 formed a flow path from the plurality of flow holes 72d to 72g to the opening 73a. The soft portion 74 is formed by filling the gap portion 79 with a soft resin material to a position at a height whereby at least the pressure sensitive elements 71c are covered.

The opening 73a has an elliptical shape, and an inner edge 73d of the opening 73a includes an edge portion that curves with respect to the first direction corresponding to the circumferential direction (width direction) of the wrist 100 when the device is worn. The opening 73a extends through the protrusion portion 73b in the thickness direction, for example. The opening 73a has a size and shape such that all of the pressure sensitive elements 71c of the pressure sensor portion 71 can be disposed therein. In other words, in this configuration, the protrusion portion 73b does not cover the pressure sensitive elements 71c of the pressure sensor portion 71. Additionally, in the second direction, and end portion in the second direction of the inner edge 73d of the opening 73a and the pressure sensitive element arrays 71d are separated from one another. In other words, the widest portion of the central portion in the first direction of the opening 73a is located away from the pressure sensitive element arrays 71d in the second direction with distance therebetween.

The soft portion 74 is formed from a relatively soft resin material such as a silicone resin. The soft portion 74 is provided in the opening 73a of the sensor head cover 73 and protects the pressure sensitive elements 71c by covering the pressure sensor portion 71. The soft portion 74 is formed, for example, by injecting a soft resin material into the opening 73a. An end surface 74a of the soft portion 74 is formed flush with the end surface of the sensor head cover 73. Note that it is sufficient that the soft portion 74 comes into contact with the wrist 100 and is formed from a material that allows the pressure of the radial artery 110 to be detected by the pressure sensitive elements 71c, and the thickness, shape that comes into contact with the wrist 100, and material of the soft portion 74 can be selected as appropriate.

The end surface 74a, which is an exposed surface of the soft portion 74, has the same shape as the inner edge 73d of the opening 73a and has an elliptical shape elongated in the first direction in which the pressure sensitive elements 71c are arranged side by side. The soft portion 74 includes an outer edge portion 74b with a curved shape that curves with respect to the first direction. In other words, a boundary portion LB between the sensor head cover 73 and the soft portion 74 has a curved shape that curves with respect to the first direction. In addition, in the central portion of the soft portion 74 in the first direction, the length in the second direction of the soft portion 74 is greater than the length in the second direction of the region where the two pressure sensitive element arrays 71d are disposed. In other words, both end portions in the second direction of the outer edge portion 74b of the soft portion 74 are separated from the pressure sensitive element arrays 71d. In other words, the widest portion of the central portion in the first direction of the end surface 74a of the soft portion 74 is located away from the pressure sensitive element arrays 71d in the second direction with distance therebetween. The soft portion 74 is formed, inside the gap portion 79 that communicates with the opening 73a, from a soft resin material with a predetermined depth, from the opening 73a to around the circumferential wall portion 72b on the rear side of the support wall portion 72a of the sensor base 72. With this soft portion 74, the pressure sensor portion 71, the sensor base 72, and the sensor head cover 73 are adhered and fixed together and the opening 73a is sealed.

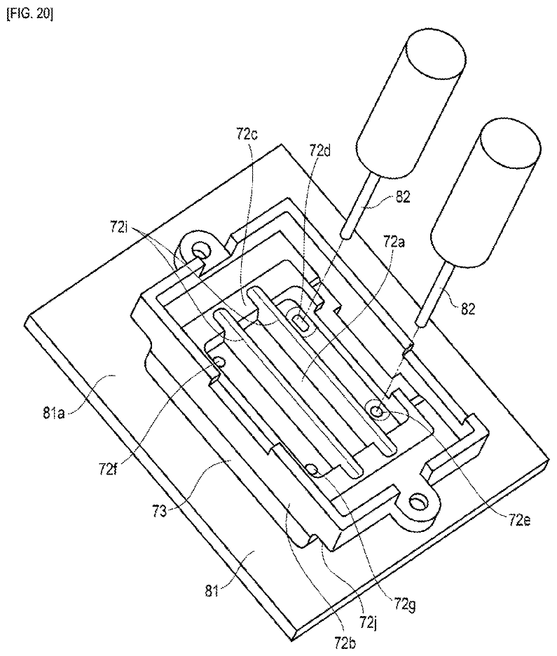

Next, a method for manufacturing the blood pressure measurement device and the sensor module will described with reference to FIGS. 13 to 21. The method for manufacturing the blood pressure measurement device and the sensor module includes: a sensor setting step of setting the pressure sensor portion 71 on the sensor base 72 (step ST1), a cover attaching step of attaching the sensor head cover 73 to the sensor base 72 (step ST2), and a filling step of supplying the soft resin with the opening 73a closed off by an opposing member (step ST3).

First, in the sensor setting step (step ST1), the pressure sensor portion 71 is set on the sensor base 72 via the adhesive sheet 71e. Specifically, first, the plurality of pressure sensitive elements 71c are mounted on the substrate 71b. Next, the substrate 71b on which the plurality of pressure sensitive elements 71c are mounted is mounted on the flexible substrate 71a. In this way, the pressure sensor portion 71 is completed. Next, the pressure sensor portion 71 is fixed on the sensor base 72 via the adhesive sheet 71e.

Then, in the cover attaching step (step ST2), the sensor head cover 73 is put on the sensor base 72. At this time, the pressure sensor portion 71 is disposed in an area corresponding to the opening 73a of the sensor head cover 73. Also, the gap portion 79 is formed between the sensor base 72 and the sensor head cover 73. The gap portion 79 forms a flow path from the opening 73a, through the opening 71f and the cutout portion 71g of the flexible substrate 71a and the adhesive sheet 71e and the flow holes 72d to 72g of the sensor base 72, to the recess portion 72c on the rear side of the sensor base 72.

In the filling step (step ST3), first, with the sensor base 72 and the sensor head cover 73 in an assembled state, the sensor base 72 and the sensor head cover 73 are disposed with the sensor head cover 73 side facing downward, opposite an opposing plate 81 (opposing member) having a smooth surface 81a, closing off the opening 73a. In this state, a nozzle 82 that discharges a soft resin material is inserted into the first hole 72d and the second hole 72e, and the soft resin material is supplied via the first hole 72d and the second hole 72e. For example, due to its own weight, the soft resin material flows from the first hole 72d into the gap portion 79 and fills up the gap portion 79 to a position at a predetermined height covering the pressure sensor portion 71. In this manner, the soft resin material is disposed in the opening 73a. At this time, the other holes 72f, 72g are flow outlets for discharging air and excess amounts of the resin material.

The soft portion 74 is formed by cooling or heating the soft resin material disposed in the gap portion 79 including the opening 73a, depending on the type of soft resin material, and solidifying the soft resin material. The sensor base 72, a support plate 77, a circuit board 78, and the sensor head cover 73 are adhered and fixed together by the soft portion 74. After the soft portion 74 is formed, the opposing plate 81 is removed at a predetermined timing. After the soft portion 74 is formed and the opposing plate 81 is removed, the soft portion 74 may be subjected to surface treatment further. In this manner, the sensor module 63 is completed.

With the blood pressure measurement device 1 worn on the wrist 100, the movable base 64 is supported in the movable case 61 in a manner allowing the movable base 64 to be movable in a direction toward and a direction away from the wrist 100. For example, the movable base 64 is capable of moving along the plurality of cylindrical members provided in the movable case 61, for example. The end portion of the movable base 64 on the wrist 100 side is fixed to the sensor base 72. Thus, the movable base 64 supports the sensor base 72 in a manner allowing the sensor base 72 to be movable in one direction with respect to the movable case 61.

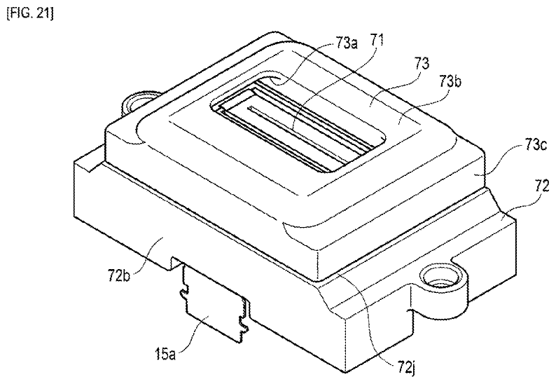

As illustrated in FIG. 22, the adjustment portion 53 is configured to adjust the position of the sensor unit 52, with respect to the case 51, in the circumferential direction of the wrist 100. The adjustment portion 53 is located on the outer surface of the case 51 and includes an adjustment catch 53a, the portion of which is fixed to the fixing portion 61b of the movable case 61 via the third hole portion 51e. Also, the adjustment portion 53 includes: graduations 53b provided adjacent to the third hole portion 51e of the case 51 and an instruction portion 53c provided on the adjustment catch 53a that indicates the graduations 53b.

The adjustment catch 53a is connected to the sensor unit 52 by being fixed to the movable case 61. The adjustment catch 53a is configured to move the sensor unit 52. In other words, the adjustment portion 53 is an adjustment mechanism that, by the adjustment catch 53a being moved in the longitudinal direction of the third hole portion 51e, moves the sensor unit 52 along the second groove 51f2 and adjusts the position of the sensor unit 52 with respect to the case 51.

The graduations 53b and the instruction portion 53c are display portions that display the position of the adjustment catch 53a, i.e., the position of the sensor unit 52 connected to the adjustment catch 53a, in a visually recognizable manner.

The fastener 43 includes, for example, one or a plurality of band-like bands and a fixing member such as a hook-and-loop fastener that secures the band wrapped around the wrist 100. The fastener 43 fixes the attach portion 41 and the sensing body 42 to the wrist 100. Note that the fastener 43 may be composed of: a first belt referred to as a parent that includes a buckle; and a second belt referred to as a pointed end that is fixed to the buckle. Also, the fastener 43 may further have a configuration in which the case 51 is fixed to the attach portion 41 by the fastener 43 being wrapped around the case 51.

In other words, the fastener 43 is configured to prevent the case 51 from moving in a direction away from the attach portion 41 when the reaction force, when the sensor module 63 presses against the wrist 100 due to the expansion of the air bag 62, acts on the movable case 61 and when the case 51 is directly pressed by the movable case 61 or indirectly pressed via the adjustment catch 53a from the movable case 61.

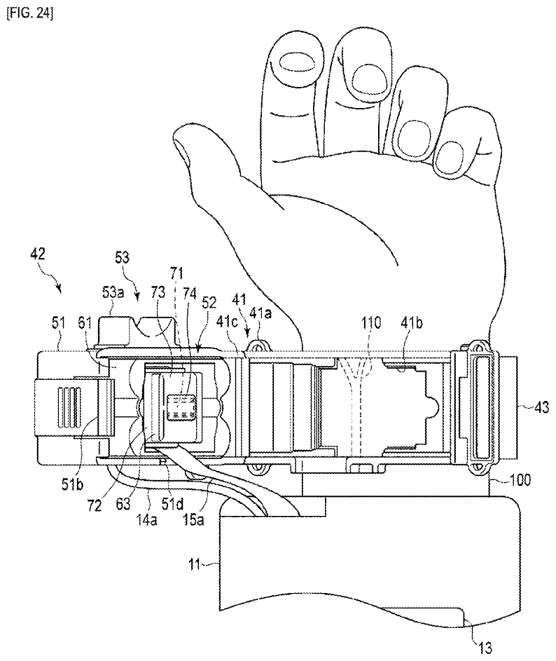

Next, an example of measurement of a blood pressure value using the blood pressure measurement device 1 will be described using FIGS. 22 to 25. FIG. 22 is an explanatory diagram illustrating an example of a blood pressure measurement using the blood pressure measurement device 1, illustrating both the operation of the user and the operation of the control unit 33. FIGS. 24 to 25 are explanatory diagrams illustrating an example of blood pressure measurement using the blood pressure measurement device 1.

First, the user searches by palpating the wrist 100 for the position of the radial artery 110 (step ST11). For example, at this time, a visible line may be drawn on the skin above the radial artery 110 with a pen.

The user then separates the sensing body 42 of the sensor device 5 from the attach portion 41. In the present embodiment, the user operates the engagement portion 51b to release the engagement of the case 51 with the base portion 41a and rotates the sensing body 42 about the rotation shaft 51a in a direction away from the attach portion 41.

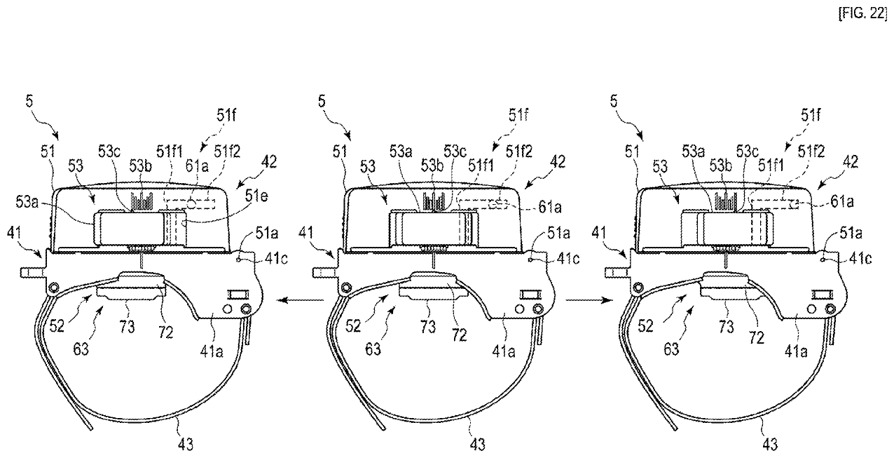

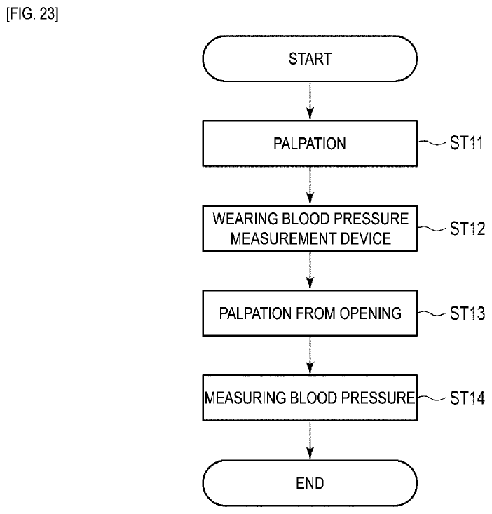

The user then attaches the device body 4 and the sensor device 5 as illustrated in FIG. 23 (step ST12). As a specific example, the user first passes the wrist 100 through the body fastener 16 of the device body 4 and the fastener 43 of the sensor device 5 and places the device body 4 and the sensor device 5 at a predetermined position on the wrist 100. Next, the body fastener 16 of the device body 4 is tightened, and the device body 4 is fixed to the wrist 100. Here, in a case of configuration in which a cuff is provided on the body fastener 16 of the device body 4, a check is performed to see whether the skin of the wrist 100 is caught in the body fastener 16 (cuff) and whether the body fastener 16 (cuff) is too loose is performed. Next, the position of the sensor device 5 is adjusted so that the opening portion 41b of the attach portion 41 of the sensor device 5 is located at the radial artery 110 of the wrist 100. In addition, the user tightens the fastener 43 of the sensor device 5, and the sensor device 5 is fixed to the wrist 100, with the radial artery 110 held at the position of the opening portion 41b.

Next, as illustrated in FIG. 24, the user palpates the wrist 100 from the opening portion 41b of the attach portion 41 (step ST13) and checks again that the radial artery 110 is located at the opening portion 41b. Then, as illustrated in FIG. 25, the user rotates the sensing body 42 in a direction toward the attach portion 41 and fixes the sensing body 42 to the attach portion 41 via the engagement portion 51b. Note that when the position of the sensing body 42 is misaligned with the radial artery 110, the adjustment catch 53a is operated to adjust the position of the sensing body 42.

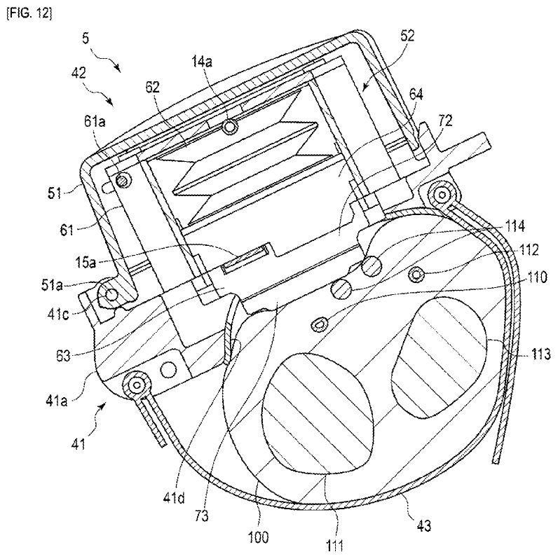

Next, the user operates the operation portion 12 to send an instruction to measure the blood pressure. The control unit 33 measures the blood pressure on the basis of the blood pressure measurement instruction (step ST14). At this time, the control unit 33 drives and controls the pump 14, and, as illustrated in FIGS. 7 to 12, the air bag 62 is expanded, moving the sensor module 63 progressively toward the wrist 100 from a state of being housed inside the movable case 61, and the sensor head cover 73 and the soft portion 74 of the sensor module 63 are pressed against the region where the radial artery 110 of the wrist 100 is found, as illustrated in FIGS. 9 and 12. By pressing the sensor head cover 73 and the soft portion 74 against this region of the wrist 100, the radial artery 110 is pressed with an appropriate amount of pressure so that a portion of the radial artery 110 is flattened, as illustrated in FIG. 12. In this state, the pressure sensitive elements 71c of the pressure sensor portion 71 measure the pressure pulse waves.

Note that the control unit 33 obtains the blood pressure via the tonometry method from the pressure pulse waves of the radial artery 110 detected by the pressure sensor portion 71. Note that prior to blood pressure measurement, the control unit 33 may perform a blood pressure measurement for calibration on the basis of program data stored in the storage unit 32 or may perform a check to determine whether or not the worn state of the device body 4 and/or the sensor device 5 and the position of the pressure sensor portion 71 are correct.

According to the blood pressure measurement device 1 of an embodiment of the present invention as described above, the end surface 74a, which is an exposed surface of the soft portion 74, has the same shape as the inner edge 73d of the opening 73a and has an elliptical shape elongated in the first direction in which the plurality of pressure sensitive elements 71c are arranged side by side. In other words, the soft portion 74 includes the outer edge portion 74b including an edge portion with a curved shape that curves with respect to the first direction. Thus, peeling off can be suppressed when sliding against the surface of the living body occurs. That is, in the case in which the sensor module 63 is misaligned in the length direction of the wrist when the blood pressure measurement device 1 is worn on the wrist, the sensor module 63 is easily moved in the second direction orthogonal to the first direction. In this case, for example, when the boundary between the soft portion and the sensor head cover extends in a linear manner in a direction orthogonal thereto, the soft portion receives a force in the direction of peeling off from the sensor head cover due to friction from movement of the living body. In contrast, in the blood pressure measurement device 1, the line of the boundary has a curved shape. Thus, the force received by the edge portion of the soft portion 74 due to friction from movement can be reduced, and separation from the sensor head cover 73 can be minimized or prevented. Thus, by preventing a gap from being formed between the soft portion 74 and the opening 73a, adhesion between the soft portion 74 and the opening 73a can be retained, waterproofing can be ensured, and dirt and the like collecting in the gap can be prevented. In addition, by preventing peeling off of the soft portion 74, negative effects on sensing accuracy can be minimized or prevented.

Also, in the second direction, the end portion in the second direction of the outer edge of the boundary and the pressure sensitive element arrays 71d are separated. With this configuration, when the sensor module 63 moves in the longitudinal direction of the living body relatively thereto, even if the soft portion 74 separates from the outer edge, because there is distance to the pressure sensitive element arrays 71d, peeling off affecting the sensing portion can be prevented.

Note that the present invention is not limited to the embodiment described above. For example, in the example described above, the external shape of the boundary portion LB is an elliptical shape. However, no such limitation is intended. For example, the boundary portion LB may have a circular shape or a configuration including an inclined side.

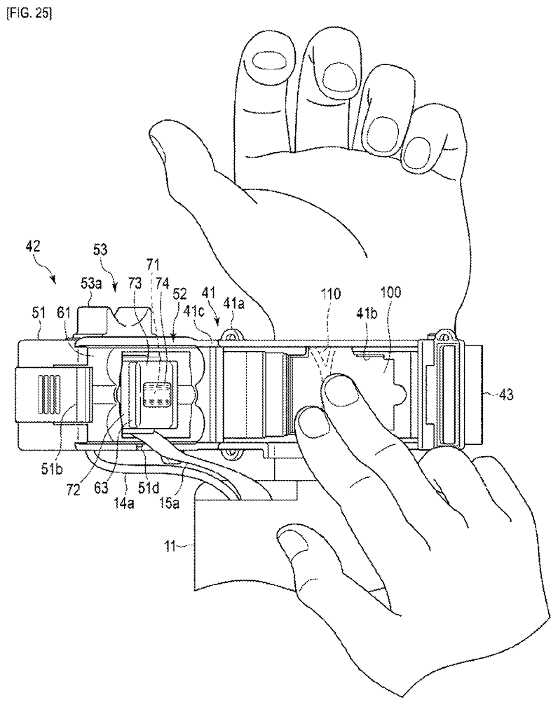

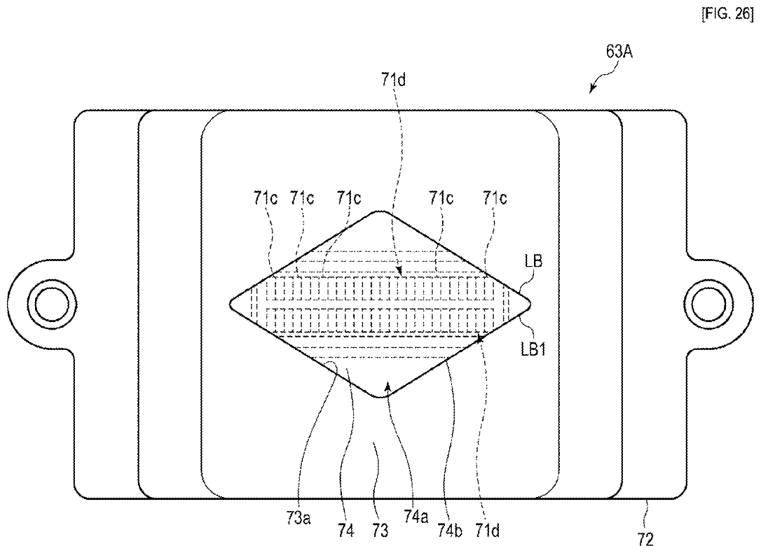

In another embodiment, a sensor module 63A illustrated in FIG. 26 has a configuration in which the external shape of the boundary portion LB, the inner edge 73d of the opening 73a, and the external shape of the end surface 74a of the soft portion 74 all have a rhombic shape. That is, the boundary portion LB includes an edge portion including four inclined sides inclined with respect to the first direction and the second direction. The four corners of the external shapes of the inner edge 73d and the end surface 74a of the soft portion 74 are rounded and formed with a curved shape. Note that configurations other than the sensor module 63A are similar to those of the sensor module 63.

In this aspect as well, similar effects to the sensor module 63 are obtained. In other words, by the line of the boundary portion LB being inclined, the resistance during movement can be dispersed, peeling off can be suppressed, waterproofing can be ensured, and dirt and the like collecting in the gap can be prevented.

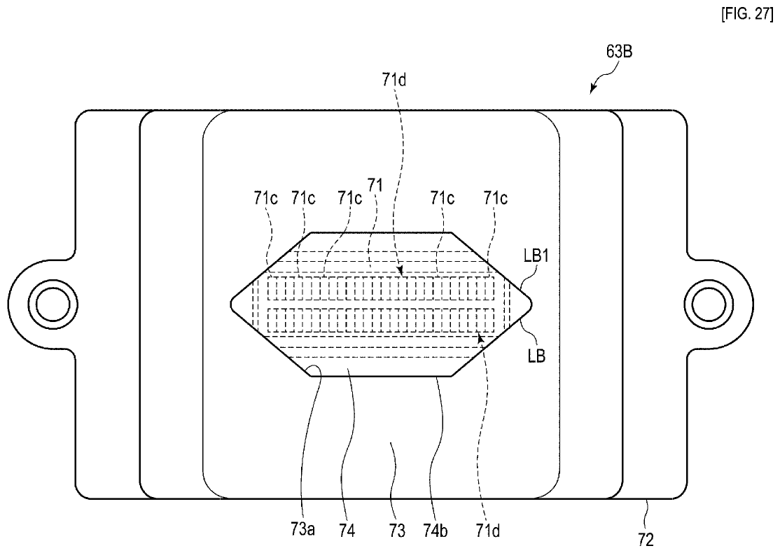

In another embodiment, a sensor module 63B illustrated in FIG. 27 has a configuration in which the external shape of the boundary portion LB, the inner edge 73d of the opening 73a, and the external shape of the end surface 74a of the soft portion 74 have a hexagonal shape. That is, the boundary portion LB decreases in width at both ends in the first direction, which is the longitudinal direction, and includes an edge portion including four inclined sides inclined with respect to the first direction and the second direction. Also, the corners of both ends in the first direction of the external shapes of the inner edge 73d and the end surface 74a of the soft portion 74 are rounded and curved. Note that configurations other than the sensor module 63B are similar to those of the sensor module 63.

In this aspect as well, similar effects to the sensor module 63 are obtained. In other words, by the line of the boundary portion LB being inclined, the resistance during movement can be dispersed, peeling off can be suppressed, waterproofing can be ensured, and dirt and the like collecting in the gap can be prevented.

Additionally, for example, in another embodiment, as illustrated by a sensor module 63C in FIG. 28, the external shape of the boundary portion LB may include inclined sides. The sensor module 63C has a configuration in which the external shape of the inner edge 73d of the opening 73a and the end surface 74a of the soft portion 74 have increased in width at both ends in the first direction and have a tapered shape. That is, the external shape of the boundary portion LB includes, at both ends in the first direction, which is the longitudinal direction, an edge portion including four inclined sides inclined with respect to the first direction and the second direction. Note that configurations other than the sensor module 63C are similar to those of the sensor module 63.

In this aspect as well, similar effects to the sensor module 63 are obtained. In other words, by the line of the boundary portion LB being inclined, the resistance during movement can be dispersed, peeling off can be suppressed, waterproofing can be ensured, and dirt and the like collecting in the gap can be prevented.

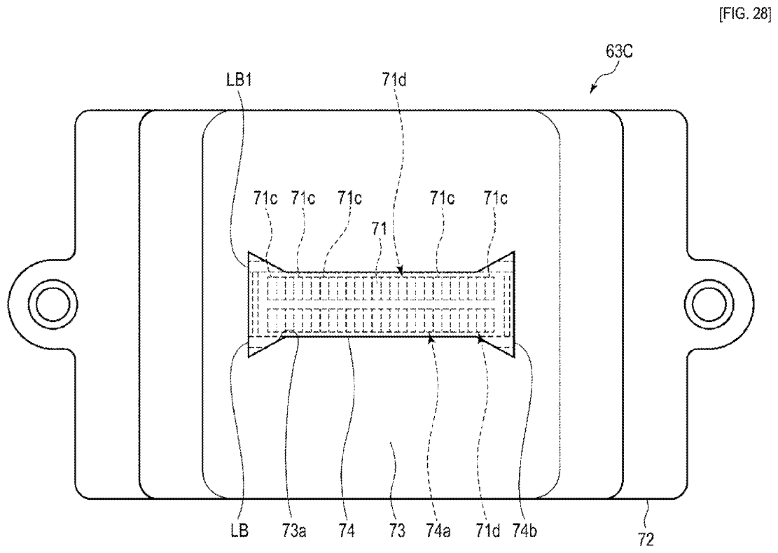

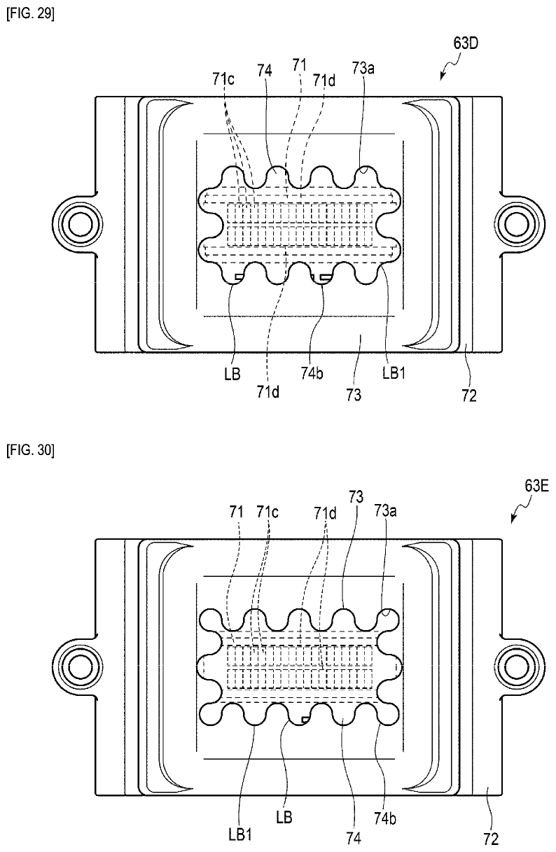

Additionally, for example, in other embodiments, such as illustrated by sensor modules 63D, 63E in FIGS. 29 and 30, the external shape of the boundary portion LB may include a wave-shaped edge portion. The sensor modules 63D, 63E have a configuration in which a portion of the inner edge 73d of the opening 73a and a portion of the outer edge portion 74b of the end surface 74a of the soft portion 74 have a wave shape. That is, the external shape of the boundary portion LB includes, at both ends in the first direction, which is the longitudinal direction, an edge portion that curves in the first direction and the second direction. Note that configurations other than the sensor modules 63D, 63E are similar to those of the sensor module 63.

In this aspect as well, similar effects to the sensor module 63 are obtained. In other words, by the line of the boundary portion LB being curved, the resistance during movement can be dispersed, peeling off can be suppressed, waterproofing can be ensured, and dirt and the like collecting in the gap can be prevented. Furthermore, with the wave shape, the distance of the edge portion is increased and the adhesion region is increased. Thus, the adhesion between the sensor head cover 73 and the soft portion 74 can be increased, and a configuration more resistant to peeling off can be achieved.

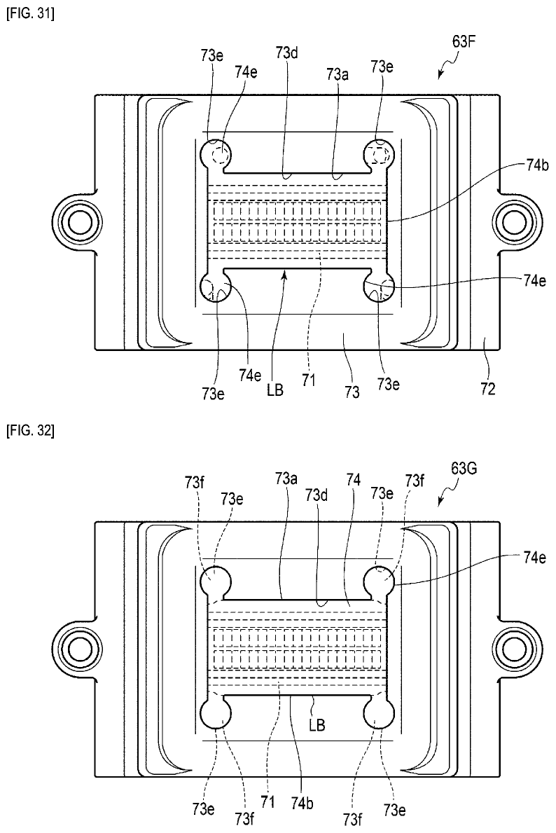

Additionally, for example, in another embodiment, such as illustrated by a sensor module 63F in FIG. 31, the boundary portion LB may have a configuration including a circular engagement portion LB1 at the corner portion that projects toward the outer circumference side. In the sensor module 63F, the inner edge 73d of the opening 73a of the sensor head cover 73 has a rectangular shape, and in each of the four corners thereof, an engagement hole 73e having a circular shape is formed. The soft portion 74 is formed so as to correspond to the opening 73a, and at each of the corner portions, an engagement projection 74e that is outwardly bulging and having a circular shape is formed. The engagement hole 73e and the engagement projection 74e together form a keyhole shape in which the corner portions bulge into a circular shape, and the base end portion of the engagement hole 73e is configured to fit into the base end portion of the engagement projection 74e. With this configuration, the engagement projection 74e is resistant to falling out of the engagement hole 73e. Note that configurations other than the sensor module 63F are similar to those of the sensor module 63.

In this aspect as well, similar effects to the sensor module 63 are obtained. That is, because the boundary portion LB includes the circular engagement portion LB1 at the corner portions, the engagement projection 74e can be prevented from falling out of the engagement hole 73e, and peeling off between the soft portion 74 and the sensor head cover 73 can be prevented.

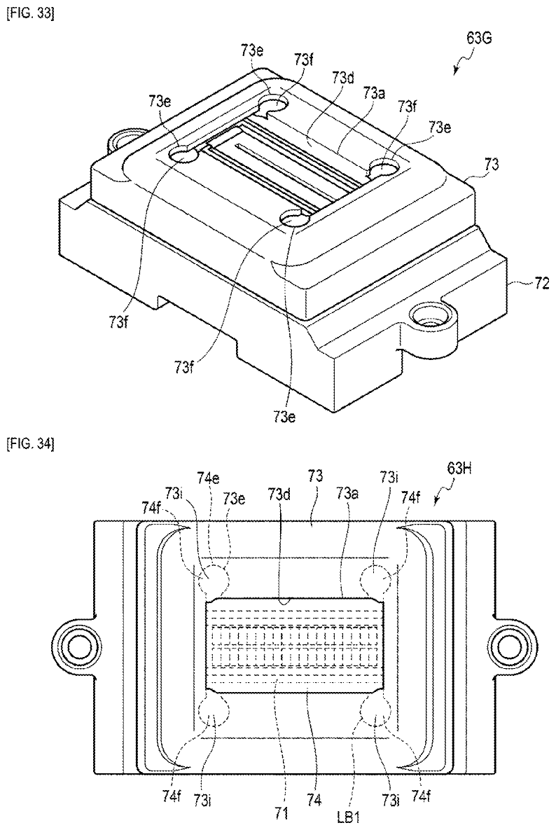

Furthermore, in the embodiments described above, the shape of the opening 73a and the soft portion 74 are uniform in the extend-through direction. However, no such limitation is intended. FIGS. 32 and 33 are a plan view and a perspective view illustrating the configuration of a sensor module 63G according to another embodiment. FIG. 33 illustrates the shape of the sensor head cover 73 before the soft portion 74 is formed. As illustrated in FIGS. 32 and 33, the end surface of the sensor module 63G on one side of the sensor head cover 73 includes a step portion 73f at each of the corner portions that is recessed to the other side away from the living body. The surface of the step portion 73f on one side is covered by the engagement projection 74e of the soft portion 74. In other words, the shape of the opening 73a is different in the depth direction, on one end side, which is the top surface side, includes the engagement hole 73e bulging outward at the corner portion, and, on the other end side, includes a rectangular shape with no engagement holes 73e. The shape of the outer edge of the soft portion 74 also differs between the one end side and the other end side. In other words, the soft portion 74 is formed with the engagement projection 74e including a predetermined thickness corresponding to the step portion 73f of the opening 73a on the top surface side, and the soft portion 74 is formed, from a predetermined depth on the other end side, with a rectangular shape with no engagement projections 74e. Note that configurations other than the sensor module 63G are similar to those of the sensor module 63F.

In this aspect as well, similar effects to the sensor module 63F are obtained. That is, because the boundary portion LB includes the circular engagement portion LB1 that bulges increasing in width from the corner portion outward, the engagement projection 74e can be prevented from falling out of the engagement hole 73e, and peeling off between the soft portion 74 and the sensor head cover 73 can be prevented.

Additionally, because the opening 73a of the sensor head cover 73 is provided with the step portion 73f, the load acting on the engagement projection 74e from the living body side is received by the step portion 73f. Thus, the soft portion 74 deforming as a whole can be suppressed, effects on the sensing portion can be minimized or prevented, and the amount of soft resin material can be reduced.

FIG. 34 is a plan view illustrating the configuration of a sensor module 63H according to another embodiment. As illustrated in FIG. 34, in the sensor module 63H, a portion of the end surface 74a of the soft portion 74 on one side includes a step portion 74f at each of the corner portions that is recessed to the other side away from the living body. The surface of the step portion 74f on the one side is covered by a portion of the sensor head cover 73. That is, in the sensor module 63H, the sensor head cover 73 includes a wall portion 73i that covers a portion of the soft portion 74 at the circumferential edge portion of the opening 73a.

In other words, the shape of the soft portion 74 is different between one end side and the other end side, having a rectangular shape with no engagement projections 74e from the top surface to a predetermined depth, while, on the other side from the predetermined depth, having a polygonal shape including the engagement projection 74e bulging and increasing in width outward at the corner portions. In addition, the shape of the opening 73a of the sensor head cover 73 differs in the depth direction, on the top surface side, the wall portion 73i is formed with a predetermined thickness corresponding to the step portion 74f of the soft portion 74, and on the other side from the predetermined depth, the shape of the opening 73a is configured to have a polygonal shape including the engagement hole 73e bulging outward at the corner portions.

Note that configurations other than the sensor module 63H are similar to those of the sensor module 63F.

In this aspect as well, similar effects to the sensor module 63F are obtained. That is, because the boundary portion LB includes the circular engagement portion LB1 at the corner portions and the surface of the soft portion 74 is covered by the wall portion 73i, the engagement projection 74e can be prevented from falling out of the engagement hole 73e, and peeling off between the soft portion 74 and the sensor head cover 73 can be further prevented.

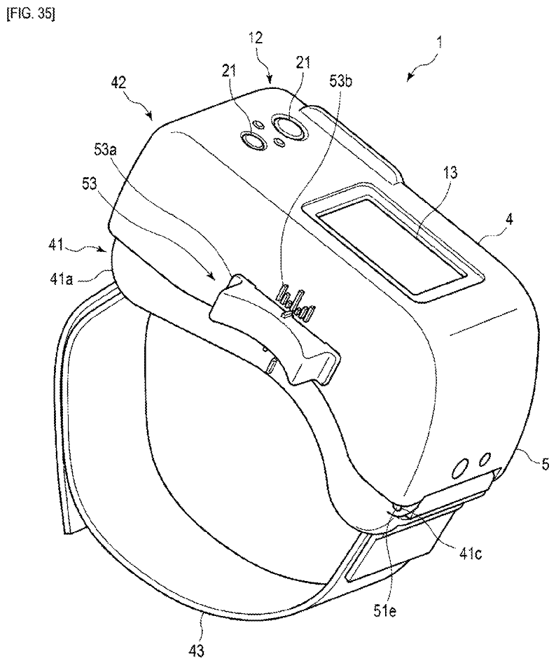

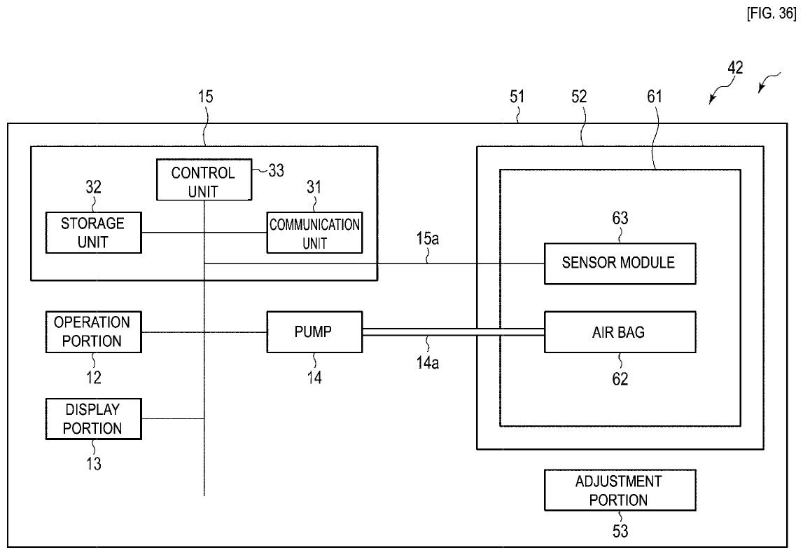

In the example described above, the blood pressure measurement device 1 has a configuration in which the device body 4 and the sensor device 5 are different bodies. However, no such limitation is intended. For example, as illustrated in FIGS. 35 and 36, the blood pressure measurement device 1 may have a configuration in which the device body 4 and the sensor device 5 are integrally formed. The blood pressure measurement device 1 with such a configuration, for example, may have configuration in which the operation portion 12, the display portion 13, the pump 14, and the control board 15 used in the device body 4 are provided in the case 51 of the sensing body 42.

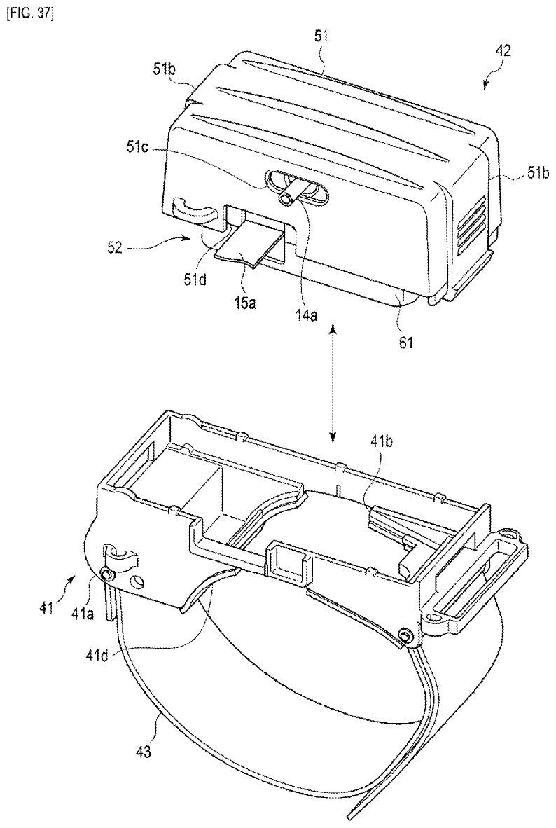

Also, in the example described above, the blood pressure measurement device 1 has a configuration in which the sensing body 42 moves in a direction away and a direction toward the attach portion 41 by the sensing body 42 rotating with respect to the attach portion 41 about an axis. However, no such limitation is intended. For example, as illustrated in FIG. 37, the blood pressure measurement device 1 may have a configuration in which the sensing body 42 moves in a direction away and a direction toward the attach portion 41 by the attach portion 41 and the sensing body 42 being separated. In the case in which the blood pressure measurement device 1 has this configuration, the engagement portions 51b are provided at a plurality of positions on the case 51 of the sensing body 42, and the sensing body 42 engages with the attach portion 41 at these positions.

Furthermore, for example, a configuration in which the sensing body 42 is configured to rotate with respect to the attach portion 41 about a single axis is also not limited to the example described above. In other words, the example described above has a configuration in which the sensing body 42 rotates with respect to the attach portion 41 about a single axis extending in a direction orthogonal to the circumferential direction of the wrist 100. However, no such limitation is intended. In other words, the sensing body 42 may rotate with respect to the attach portion 41 about a single axis extending in the direction of a tangent line to the circumferential direction of the wrist 100.

Also, in the examples described above, the blood pressure measurement device 1 measures the pressure of the radial artery 110 and obtains the blood pressure by the tonometry method. However, no such limitation is intended. In another example, the pressure of the ulnar artery is measured. The blood pressure measurement device 1 may also have a configuration in which the blood pressure is obtains via a method other than the tonometry method.

In the examples described above, the opening portion 41b of the attach portion 41 has a shape that allows for palpation of the wrist 100. However, no such limitation is intended. That is, for example, the opening portion 41b of the attach portion 41 may have a shape that allows the sensor unit 52 to come into contact with wrist 100 beyond the opening portion 41b, in a range in which position is adjusted by the adjustment portion 53.

In addition, similar to how the pressure sensitive element arrays 71d are retained on the flexible substrate 71a in the examples described above, the present invention is not limited to a device for measuring blood pressure and may be applied to other devices using other measurement methods such as devices for measuring pulse waves.

Also, in the example described above, the sensor unit 52 has a configuration in which the sensor base 72 of the sensor module 63 is supported by the movable base 64 in a manner allowing the sensor base 72 to move within the movable case 61. However, no such limitation is intended. For example, the movable base 64 may be integrally formed with the sensor base 72 of the sensor module 63.

In the example described above, the pressure sensor portion 71 is mounted on the sensor base 72. However, the present invention is not limited to this configuration. For example, the pressure sensor portion 71 may be directly fixed to the circuit board. In this case, for example, a soft portion is formed in the opening 73a of the sensor head cover 73 by attaching the sensor head cover 73 to cover the pressure sensor portion 71 and injecting a soft resin material from the rear surface of the circuit board via a flow hole formed in the circuit board.

Furthermore, the method for forming the soft portion 74 is not limited to the methods described above. For example, the soft portion 74 formed with a predetermined shape in advance may be installed in the opening 73a. In other words, in this configuration, the sensor head cover 73 is attached to cover the pressure sensor portion 71, and the soft portion 74 such as a sensor window or the like made of transparent silicone rubber, for example, formed in a predetermined shape in advance is fit into the opening 73a of the sensor head cover 73.

In other words, the embodiments described above are merely examples of the present invention in all respects. Of course, various modifications and variations can be made without departing from the scope of the present invention. Thus, specific configurations in accordance with an embodiment may be adopted as appropriate at the time of carrying out the present invention.

### REFERENCE SIGNS LIST

1 Blood pressure measurement device
4 Device body
5 Sensor device
11 Body case
12 Operation portion
13 Display portion
14 Pump
14a Tube
15 Control board
15a Cable
16 Body fastener
21 Button
31 Communication unit
32 Storage unit
33 Control unit
41 Attach portion
41a Base portion
41b Opening portion
41c Attachment portion
42 Sensing body
43 Fastener
51 Case
51a Rotation shaft
51b Engagement portion
51c Hole portion
51d Hole portion
51e Hole portion
51f Guide groove
51f1 Groove
51f2 Groove
52 Sensor unit
53 Adjustment portion
53b Graduations
53c Instruction portion
61 Movable case
61a Guide projection
61b Fixing portion
62 Air bag
63 Sensor module
71 Pressure sensor portion
71a Flexible substrate
71b Substrate
71c Pressure sensitive element
71d Pressure sensitive element array
71e Adhesive sheet
71f Opening
71g Cutout portion
72 Sensor base
72a Support wall portion
72b Circumferential wall portion
72c Recess portion
72d to 72h Flow hole (first hole, second hole, third hole, fourth hole, fifth hole)
72i Ridge portion
72j Step portion
73 Sensor head cover
73a Opening
73b Protrusion portion
73d Inner edge
73e Engagement hole
73f Step portion
73g Inclined surface
73h Corner portion
73i Wall portion
74 Soft portion
74a End surface
74b Outer edge portion
74e Engagement projection
81 Opposing plate
81a Smooth surface
82 Nozzle
100 Wrist
110 Radial artery
111 Radius
112 Ulnar artery
113 Ulna
114 Tendon

1. A sensor module, comprising:
a sensor portion;
a sensor head cover including an opening at a position opposite the sensor portion; and
a soft portion that covers the sensor portion and is disposed in the opening of the sensor head cover, a boundary portion between the soft portion and the sensor head cover including an outer edge including an edge portion that is inclined or curved, when the sensor module is worn on a living body, with respect to a first direction along a circumferential direction of the living body or a second direction along a longitudinal direction of the living body.

2. A sensor module, comprising:
a sensor portion;
a sensor base that supports the sensor portion;
a sensor head cover that is disposed opposite the sensor base and includes an opening at a position opposite the sensor portion; and
a soft portion that covers the sensor portion and is disposed in the opening of the sensor head cover, a boundary portion between the soft portion and the sensor head cover including an outer edge including an edge portion that is inclined or curved, when the sensor module is worn on a living body, with respect to a first direction along a circumferential direction of the living body or a second direction along a longitudinal direction of the living body.

3. The sensor module according to claim 1, wherein the outer edge of the soft portion is elliptical.

4. The sensor module according to claim 1, wherein the edge portion is inclined with respect to the first direction and the second direction.

5. The sensor module according to claim 1, wherein the edge portion has a wave-like shape.

6. The sensor module according to claim 1, wherein the boundary portion has a polygonal shape including an engagement portion increasing in width from a corner portion outward.

7. A sensor module, comprising:
a sensor portion;
a sensor base that supports the sensor portion;
a sensor head cover that is disposed opposite the sensor base and includes an opening at a position opposite the sensor portion; and
a soft portion disposed in the opening of the sensor head cover, the soft portion having a polygonal shape that covers the sensor portion and including an engagement projection increasing in width from a corner portion outward.

8. The sensor module according to claim 1, wherein
the sensor head cover is provided with a step portion in a circumferential edge portion of the opening at an end surface on one side, the step portion being recessed toward the other side; and
a surface of the step portion on the one side is covered by the soft portion.

9. The sensor module according to claim 1, wherein
the sensor head cover is provided, at a circumferential edge portion of the opening, with a wall portion that covers one side of the soft portion.

10. The sensor module according to claim 1, wherein
the sensor portion is provided with a pressure sensitive element array in which a plurality of pressure sensitive elements are arranged in the first direction; and
an end portion in the second direction of the outer edge of the boundary portion and the pressure sensitive element array are separated in the second direction that intersects the first direction.

11. The sensor module according to claim 2, wherein
the sensor base includes:
a pressure sensitive element array including a plurality of pressure sensitive elements disposed on one main surface side and
a support wall portion in which a flow hole is disposed in an outer circumference of a region where the pressure sensitive element array is disposed, the flow hole extending through from the one main surface side to an other main surface side;
the sensor head cover is disposed on the one main surface side of the support wall portion of the sensor base via a gap that communicates with the flow hole and the opening; and
the soft portion is formed in the opening.

12. A blood pressure measurement device, comprising:
the sensor module according to claim 1;
a sensing body including a case that houses the sensor module;
an attach portion including:
an opening portion disposed at a position opposite a region where one artery of a wrist is present, the opening portion having a shape that allows the wrist to be palpated and
an end surface that curves conforming to a portion of a shape in the circumferential direction of the wrist; and
a fastener provided on the attach portion that extends in a first direction and is wrapped around in the circumferential direction of the wrist.

13. The sensor module according to claim 2, wherein the outer edge of the soft portion is elliptical.

14. The sensor module according to claim 2, wherein the edge portion is inclined with respect to the first direction and the second direction.

15. The sensor module according to claim 2, wherein the edge portion has a wave-like shape.

16. The sensor module according to claim 2, wherein the boundary portion has a polygonal shape including an engagement portion increasing in width from a corner portion outward.

17. The sensor module according to claim 2, wherein
the sensor head cover is provided with a step portion in a circumferential edge portion of the opening at an end surface on one side, the step portion being recessed toward the other side; and
a surface of the step portion on the one side is covered by the soft portion.

18. The sensor module according to claim 3, wherein
the sensor head cover is provided with a step portion in a circumferential edge portion of the opening at an end surface on one side, the step portion being recessed toward the other side; and
a surface of the step portion on the one side is covered by the soft portion.

19. The sensor module according to claim 4, wherein
the sensor head cover is provided with a step portion in a circumferential edge portion of the opening at an end surface on one side, the step portion being recessed toward the other side; and
a surface of the step portion on the one side is covered by the soft portion.

20. The sensor module according to claim 5, wherein
the sensor head cover is provided with a step portion in a circumferential edge portion of the opening at an end surface on one side, the step portion being recessed toward the other side; and
a surface of the step portion on the one side is covered by the soft portion.
